(19) United States
(12) Patent Application Publication
(10) Pub. No.: US 2025/0302737 A1
(43) Pub. Date: Oct. 2, 2025

(54) POLYMERIC MICRONEEDLE ARRAYS CROSSLINKED BY PBA-DIOL COMPLEXES FOR GLUCOSE-RESPONSIVE INSULIN DELIVERY

(71) Applicant: UNIVERSITY OF NOTRE DAME DU LAC, South Bend, IN (US)

(72) Inventors: Matthew J. WEBBER, South Bend, IN (US); Zhou YE, South Bend, IN (US); Yuanhui XIANG, South Bend, IN (US)

(21) Appl. No.: 18/864,866

(22) PCT Filed: Jul. 25, 2023
(86) PCT No.: PCT/US2023/070893

§ 371 Date: Nov. 11, 2024

Related U.S. Application Data
(60) Provisional application No. 63/369,432, filed on Jul. 26, 2022.

(51) Int. Cl. A61K 9/00; A61K 38/26; A61K 38/28; A61L 31/04; A61L 31/14; C08F 20/34

(52) U.S. Cl. CPC A61K 9/0021; A61K 38/26; A61K 38/28; A61L 31/048; A61L 31/145; C08F 20/34

(57) ABSTRACT

Described herein are compositions and methods for manufacturing polymeric microneedle arrays crosslinked by phenylboronic acid (PBA)-diol complexes for glucose-responsive insulin delivery.

### CROSS-REFERENCE TO RELATED APPLICATIONS

This application claims priority to U.S. Provisional Patent Application No. 63/369,432, filed on Jul. 26, 2022, which is incorporated by reference herein in its entirety.

### BACKGROUND

With a rapid increase in prevalence, diabetes is a disease in need of improved therapeutic approaches. Current therapeutic management for many with diabetes, and particularly those with Type-1 diabetes, entails repeated daily insulin self-administration and blood glucose monitoring to regulate blood glucose. Yet, insulin administration is complicated by the need for dose estimation and adjustment, with incorrect dosing causing an assortment of both acute and chronic health complications. As such, strategies that enable more convenient and autonomous blood glucose control are an area of active exploration, and many approaches are being evaluated to prepare glucose-responsive insulin therapies wherein the bioavailability or potency of insulin is dictated by blood glucose level. One commonly explored approach to glucose-responsive therapies has used dynamic-covalent bonding between phenylboronic acids (PBAs) and cis-1,2 or cis-1,3 diols. The dynamic PBA-diol bond is susceptible to competition from glucose (itself a cis-1,2 diol), affording glucose-dependent equilibrium bonding interactions in materials and devices used for insulin delivery.

Accordingly, PBA chemistry has been explored for glucose-responsive PBA-modified insulin variants, injectable polymer networks prepared from PBA-diol crosslinking, or responsive nanoscale excipients that change form upon glucose binding to PBA motifs.

As the skin is the largest and most accessible organ in the body, dermal delivery of therapies holds promise to prepare more convenient and painless self-administered therapeutic platforms. Microneedle arrays are particularly appealing given that their dimensions enable penetration of the protective dermal barrier without reaching depths sufficient to cause pain. Accordingly, such technologies have been of particular interest for the therapeutic delivery of proteins; gastric protein instability often precludes delivery by more convenient oral routes and therefore would otherwise require injection. Given these benefits, microneedles have been explored in the context of insulin delivery. Various glucose-sensing mechanisms have demonstrated function in this regard, with PBA-containing polymers that bind glucose to alter electrostatic repulsion and microneedle swelling being the most extensively explored. In these covalently crosslinked polymer networks, swelling arises upon PBA-glucose interaction due to the glucose-stabilized negative charge state of the boronate, and the ensuing increase in microneedle swelling drives accelerated release of insulin trapped within the polymer network. However, such microneedles are typically prepared using in situ covalent polymerization techniques that yield non-degradable chemically crosslinked polymer networks prepared within microneedle molds. Such a process introduces risks for physiological exposure to toxic unreacted monomers, crosslinkers, or initiators of polymerization.

What is needed are polymeric microneedle arrays crosslinked by PBD-diol complexes for glucose-responsive insulin delivery.

### SUMMARY

One embodiment described herein is a polymer comprising:
(i) recurring units of formula (I):

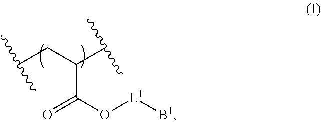

wherein:
L¹ is a linking moiety; and
B¹ is

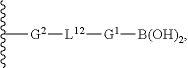

wherein:
G¹ is a pyridylene or a phenylene, wherein G¹ is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₃₋₄ cycloalkyl, —NO₂, or —OC₁₋₂ haloalkyl;
L¹² is a C₁₋₆ alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and
G² is phenylene, wherein G² is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₁₋₂ haloalkyl, —OC₃₋₄ cycloalkyl, or —NO₂; and
(ii) recurring units of formula (II)

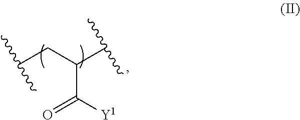

wherein:
Y¹ is —NR^{x}R^{y} or —OR^{x}; and R^{x} and R^{y} are each independently hydrogen, C₁₋₄ alkyl, or C₃₋₄ cycloalkyl.
   In one aspect, B¹ comprises:

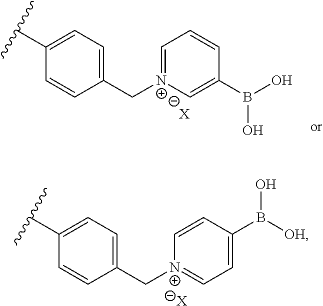

wherein: X^{Θ} is an anion having a net charge of −1. In another aspect, X^{Θ} is Br⁻, Cl⁻, NO₃⁻, H₂PO₄⁻, H₂PO₃⁻, HSO₄⁻, HSO₃⁻, H₃C—SO₃⁻, HCO₃⁻, HCO₂⁻, H₃C—CO₂, HC₂O₄⁻, or TsO⁻. In another aspect, X^{Θ} is Br or Cl⁻. In another aspect, B¹ comprises:

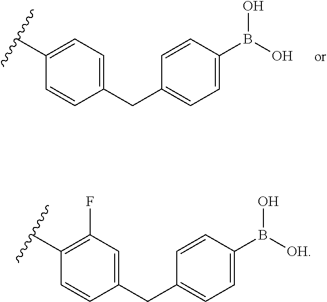 [SMILES: *c1ccc(Cc2ccc(B(O)O)cc2)cc1 ; *c1ccc(Cc2ccc(B(O)O)cc2)cc1F]

In another aspect, L¹ comprises an amide moiety.
In another aspect, L¹ comprises

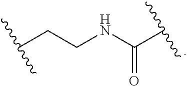 [SMILES: *CCNC(*)=O]

In another aspect, the recurring units of formula (II) are acrylamide units of formula (II-a):

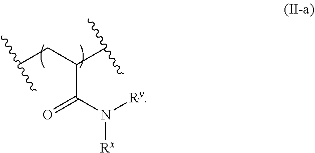

In another aspect, R^{x} and R^{y} are each methyl. In another aspect, the molar ratio of the units of formula (II) to the units of formula (I) is about 1:1 to about 20:1. In another aspect, the molar ratio of the units of formula (II) to the units of formula (I) is about 5:1. In another aspect, the polymer has a number average molecular weight (Mₙ) of about 3,000 g/mol to about 30,000 g/mol as measured by gel permeation chromatography. In another aspect, the polymer has a Mₙ of about 5,000 g/mol to about 8,500 g/mol as measured by gel permeation chromatography. In another aspect, the recurring unit of formula (I) is repeated 3 times to 50 times. In another aspect, the recurring unit of formula (II) is repeated 20 times to 100 times.

Another embodiment described herein is a hydrogel comprising a polymer described herein crosslinked with a diol crosslinker. In one aspect, the molar ratio of B¹ of the polymer to the diol of the diol crosslinker is from about 0.25:1 to about 10:1. In another aspect, insulin, an insulin variant, an insulin analogue, glucagon, GLP-1, or a combination thereof is encapsulated within the hydrogel.

Another embodiment described herein is a hydrogel comprising: a polymer described herein crosslinked with a diol crosslinker including a multi-armed polymer, wherein each individual arm comprises a diol of formula (III):

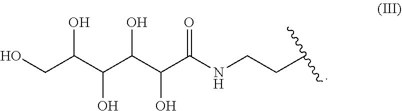 [SMILES: *CCNC(=O)C(O)C(O)C(O)C(O)CO]

In one aspect, the molar ratio of B¹ of the polymer to the diol of the diol crosslinker is from about 0.25:1 to about 10:1. In another aspect, the multi-armed polymer comprises a polyalkylene glycol. In another aspect, the multi-armed polymer is a four-armed or an eight-armed polymer.

Another embodiment described herein is a pharmaceutical composition comprising insulin, an insulin variant, an insulin analogue, glucagon, or GLP-1, or a combination thereof encapsulated within a hydrogel described herein, and a pharmaceutically acceptable excipient.

Another embodiment described herein is a device comprising: a microneedle array comprising a plurality of microneedles on a surface of a substrate, each microneedle comprising a polymer described herein crosslinked with a diol crosslinker, the diol crosslinker including: a multi-armed polymer, wherein each individual arm comprises a diol of formula (III):

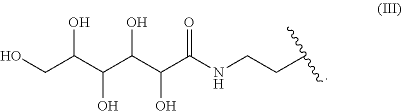 [SMILES: *CCNC(=O)C(O)C(O)C(O)C(O)CO]

wherein the molar ratio of the units of formula (II) to the units of formula (I) is about 1:1 to about 20:1, and the molar ratio of B¹ of the polymer to the diol of the diol crosslinker is about 0.25:1 to about 10:1. In one aspect, the diol crosslinker comprises:

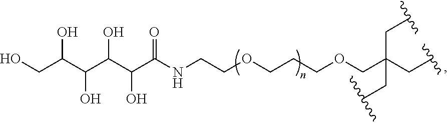

wherein n is 2 to 250. In another aspect, the molar ratio of the units of formula (II) to the units of formula (I) is about 5:1. In another aspect, the molar ratio of B¹ of the polymer to the diol of the diol crosslinker is about 4:1 to about 8:1. In another aspect, each microneedle has a length of about 300 μm to about 800 μm. In another aspect, each microneedle lacks a channel extending through the length of the microneedle. In another aspect, the substrate comprises the same polymer crosslinked with the diol crosslinker as each microneedle. In another aspect, each microneedle has a failure point of greater than 0.6 N/needle. In another aspect, each microneedle further comprises insulin, an insulin variant, an insulin analogue, glucagon, GLP-1, or a combination thereof.

Another embodiment described herein is a method of making a device comprising a microneedle array, the method comprising: adding a hydrogel described herein to a mold, the mold comprising a plurality of microneedle molds; applying a force to the hydrogel such that the hydrogel fills each microneedle mold; and drying the hydrogel to provide a device comprising a microneedle array, the microneedle array comprising a plurality of microneedles that align in number and arrangement with the plurality of microneedle molds, wherein each microneedle comprises the dehydrated hydrogel. In one aspect, the insulin, insulin variant, insulin analogue, glucagon, or GLP-1, or combination thereof is encapsulated in the hydrogel. In another aspect, the method does not include crosslinking of the hydrogel during or after adding to the mold.

Another embodiment described herein is a method of delivering insulin to a subject in need thereof, the method comprising: contacting an area of the subject's skin with the device of claim 24, wherein the insulin, insulin variant, insulin analogue, glucagon, GLP-1, or combination thereof is transdermally delivered to the subject. In one aspect, the subject has diabetes.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the hierarchical preparation of dynamic-covalent hydrogels and microneedles. A synthesized polymer bearing pendant phenylboronic acid (PBA) motifs is combined with a multivalent diol (PEG₄ₐ-Diol) to form hydrogels with dynamic-covalent PBA-Diol crosslinking susceptible to competition from glucose. Filling of these hydrogels in microneedle molds and drying affords fabrication of polymer microneedle arrays capable of delivering insulin through the skin.

FIG. 2A-C show structures and ¹H NMR spectra for the synthesis of poly(DMAA-co-PyPBA), including precursors of a reversible addition fragmentation chain-transfer polymerization (RAFT)-synthesized poly(DMAA-co-^{boc}NEA) (FIG. 2A), deprotected amine precursor poly(DMAA-co-NEA) (FIG. 2B), and the final poly(DMAA-co-PyPBA) (FIG. 2C). Proton labels correspond to peaks in the respective ¹H NMR spectra of each polymer, with integrations shown for each peak of interest.

FIG. 3 shows gel permeation chromatography of poly(DMAA-co-PyPBA) supporting a degree of polymerization of 70.

FIG. 4A-C show rheology of 7.5% (w/v) hydrogels prepared with different PyPBA:diol ratios The presence of 400 mg/dl glucose did not significantly alter the moduli in hydrogels with (FIG. 4A) 2:1 PyPBA:diol but affected the moduli in a similar manner for hydrogels with (FIG. 4B) 4:1 and (FIG. 4C) 8:1 PyPBA:diol.

FIG. 5A shows oscillatory rheology frequency sweeps of PyPBA-Diol dynamic-covalent hydrogels prepared at 5, 7.5, and 10 wt % total polymer content. Both network relaxation time (T_{R}), indicated by the G′/G″ crossover point, and the terminal regime scaling (G′∝ω², G″∝ω′) is the same across all three concentrations. The impact of different glucose levels on hydrogel properties is shown for hydrogels prepared at 5 wt % (FIG. 5B), 7.5% (FIG. 5C), and 10 wt % (FIG. 5D), with samples prepared in conditions of no glucose (PBS) or PBS with glucose concentrations of 100, 200, or 400 mg/dL. A single representative rheology trace is shown for each condition, though the observed trends were reproduced over several experiments.

FIG. 6 shows rheological frequency sweep comparing 10 wt % hydrogels prepared in PBA in the presence or absence of 5000 mg/dL glucose.

FIG. 7A-B show the rheological characterization of the hydrogels to assess (FIG. 7A) shear-thinning with a shear rate ramp under flow and (FIG. 7B) self-healing through an oscillatory step-strain study at 10 rad/s varying strain from 1% to 1000% for two cycles.

FIG. 8A-D show the fabrication of microneedles entailed (FIG. 8A) filling a preformed mold with a hydrogel prepared by PyPBA-Diol dynamic-covalent crosslinking of polymer/macromer precursors and centrifuging the hydrogel to fill the mold pattern. FIG. 8B shows following drying and peeling, an array of formed microneedles was created with uniform conical geometry and minimal defects as visualized by SEM. FIG. 8C shows the inclusion of FITC-labeled insulin the hydrogel prior to microneedle processing resulting in insulin distributed uniformly throughout the dried microneedles, visualized by fluorescent optical microscopy. FIG. 8D shows the mechanical properties of dried microneedle arrays were assessed by compression onto a flat stainless-steel plate, enabling the stiffness to be determined. SEM following testing (inset) demonstrates that microneedles buckle upon exposure to compression, but do not demonstrate critical failure.

FIG. 9 shows SEM images of microneedle arrays at lower zoom to support the effectively defect-free nature of the microneedle arrays arising from molding of dried hydrogel.

FIG. 10 shows the in vitro release of FITC-insulin in 10 wt % hydrogels (4:1 PyPBA:diol). 10 wt % hydrogels were loaded with 0.04 wt % FITC-insulin and submerged in PBS without or with 100, 200, and 400 mg/dl glucose. Aliquots were collected at predetermined time points to measure the cumulative release of FITC-insulin. n=3 for each group. Error bars are standard errors.

FIG. 11A-B show the functional performance of PBA-Diol crosslinked materials in vitro and in vivo. FIG. 11A shows the release of FITC-labeled insulin from dried material films incubated in PBS without glucose as well as that with 400 mg/dl glucose added. Accelerated release was verified by fitting the data to the Korsmeyer-Peppas equation (model) with the values obtained from this fit tabulated for each group (n=5/group). FIG. 11B shows device performance in STZ-induced diabetic Sprague Dawley rats, comparing application of the patch with and without encapsulated insulin to the skin under fasted conditions, with serial blood glucose monitoring (n=4/group).

FIG. 12 shows the mass change in dried hydrogel films submerged in PBS without or with 400 mg/dL glucose. The material was weighed at the initial time M₀ and at subsequent timepoints the excess liquid was carefully removed, and materials were reweighed with values reported as a ratio of the change in mass over the initial mass (mass ratio). n=4 for each group. Error bars are standard deviations.

FIG. 13 shows H & E-stained skin tissue after the penetration of microneedles.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group-O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆ alkyl” means a straight or branched chain hydrocarbon containing from 1 to 6 carbon atoms. The term “C₁₋₄ alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ⁻, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl).

Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl).

Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

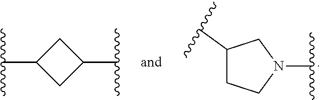 [SMILES: *C1CC(*)C1 ; *C1CCN(*)C1]

Cycloalkylene and heterocyclylene include a geminal divalent groups such as 1,1-C₃₋₆ cycloalkylene

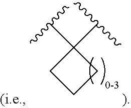

A further example is 1,1-cyclopropylene

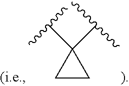 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. A monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one-OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄ alkyl,” “C₃₋₆ cycloalkyl,” or “C₁₋₄ alkylene”). These designations are used as generally understood by those skilled in the art. For example, the representation “C” followed by a subscripted number indicates the number of carbon atoms present in the group that follows. Thus, “C₃ alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄ alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The ongoing rise in diabetes incidence necessitates improved therapeutic devices to enable precise blood glucose control with convenient device form-factors. Microneedle patches are one such device platform capable of achieving therapeutic delivery through the skin. In recent years, polymeric microneedle arrays have been reported using methods of in situ polymerization and covalent crosslinking in microneedle molds. In spite of promising results, in situ polymerization carries a risk of exposure to toxic unreacted precursors remaining in the device. Here a polymeric microneedle patch is demonstrated that uses dynamic-covalent PBA-diol bonds in a dual role affording both network crosslinking and glucose sensing. By this approach, a pre-synthesized and purified polymer bearing pendant phenylboronic acid motifs is combined with a multivalent diol crosslinker to prepare dynamic-covalent hydrogel networks. The ability of these dynamic hydrogels to shear-thin and self-heal enables their loading to a microneedle mold by centrifugation. Subsequent drying then yields a patch of uniformly shaped microneedles with the requisite mechanical properties to penetrate skin. Insulin release from dried patches is accelerated in the presence of glucose. Moreover, short-term blood glucose control in a diabetic rat model following application of the device to the skin confirms insulin activity and bioavailability. Accordingly, dynamic-covalent crosslinking facilitates a route for fabricating microneedle arrays circumventing the toxicity concerns of in situ polymerization, offering a convenient device form-factor for therapeutic insulin delivery.

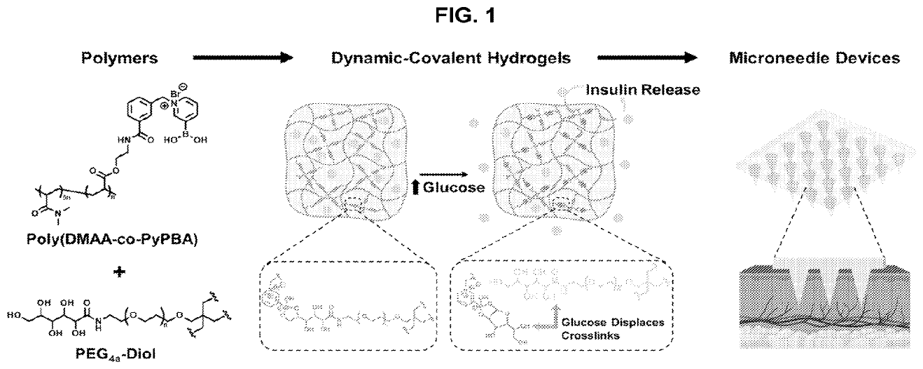

Described herein are dissolvable and glucose-responsive microneedle arrays are prepared with reversible PBA-diol complexation acting in a dual capacity as both the mechanism of hydrogel crosslinking and the trigger for glucose-responsive function. FIG. 1 depicts a pre-synthesized polymeric backbone bearing pendant PBA motifs was combined with a diol-terminated 4-arm PEG macromer to yield hydrogels capable of fabrication into microneedle arrays. The approach described here builds on existing efforts in glucose-responsive microneedles by replacing chemical covalent crosslinking with PBA-diol dynamic-covalent crosslinking. Moreover, compared to other PBA-based microneedles reported to date that are prepared by in situ polymerization in microneedle molds, the present approach enables polymer synthesis and purification prior to microneedle fabrication, circumventing risks arising from exposure to toxic monomers or initiators. Routes of fabricating glucose-responsive polymeric microneedle arrays for insulin delivery are described herein.

### Compositions

### A. Polymers

Described herein are polymers for improved glucose-responsive delivery of insulin, insulin variants, insulin analogues, glucagon, GLP-1, or combinations thereof. In various instances, the polymers comprise a pendant boronic acid moiety, referred to as “B¹.” In various instances, B¹ comprises a boronic acid, e.g., a pyridinyl or phenyl boronic acid.

In various instances, B¹ is a binding group. As used herein, “binding group” is a moiety that is capable of binding another molecule, and in some instances specifically binding another molecule. As used herein, the term “specifically binds” is generally meant that a molecule binds to a target molecule when it binds to that target molecule more readily than it would bind to a random, unrelated target. In some instances, specific binding of a molecule to a target molecule can result in a covalent bond between the molecule and the target molecule, such as a dynamic covalent bond. B¹ can bind to diol containing molecules, such as molecules having cis-1,2 diols (e.g., glucose) or cis-1,3 diols. In some instances, B¹ can specifically bind to diol containing molecules. In various instances, B¹ is a glucose binding group. In some instances, B¹ specifically binds glucose.

In various instances, the polymer comprising B¹ may be a copolymer. Suitable copolymers may include random copolymers, block copolymers, and combinations thereof. In various instances, the polymer may comprise recurring units of formula (I) and recurring units of formula (II). Various aspects of exemplary recurring units of formula (I) and exemplary recurring units of formula (II) are described below.

i

### 1. Recurring Units of Formula (I)

In various instances, the polymer comprising B¹ may comprise recurring units of formula (I):

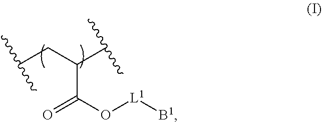

wherein: L¹ is a linking moiety; and B¹ is

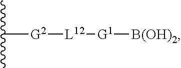

wherein: G¹ is a pyridylene or a phenylene, wherein G¹ is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₃₋₄ cycloalkyl, —NO₂, or —OC₁₋₂ haloalkyl; L¹² is a C₁₋₆ alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and G² is phenylene, wherein G² is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₁₋₂ haloalkyl, —OC₃₋₄ cycloalkyl, or —NO₂.

In some instances, B¹ may comprise:

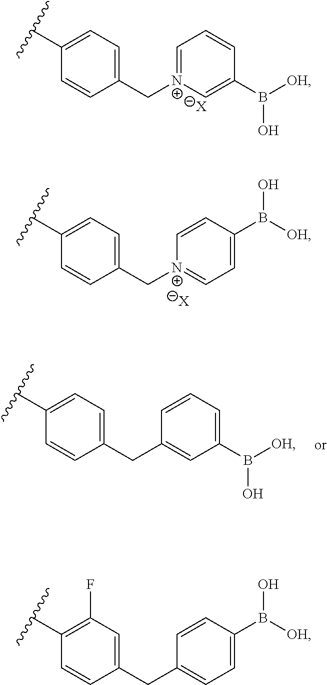

wherein X° is an anion having a net charge of −1.

In some instances, B¹ may comprise:

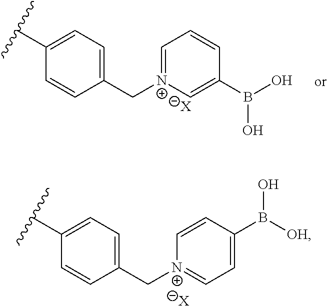

wherein X^{Θ} is an anion having a net charge of −1. In some instances, X^{Θ} may be Br⁻, Cl⁻, NO₃⁻, H₂PO₄⁻, H₂PO₃⁻, HSO₄⁻, HSO₃⁻, H₃C—SO₃⁻, HCO₃⁻, HCO₂, H₃C—CO₂⁻, HC₂O₄⁻, or TsO⁻. For example, X^{Θ} may be Br⁻ or Cl⁻. In some instances, B¹ may comprise:

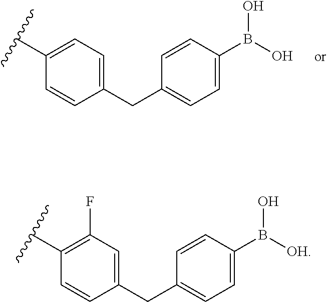 [SMILES: *c1ccc(Cc2ccc(B(O)O)cc2)cc1 ; *c1ccc(Cc2ccc(B(O)O)cc2)cc1F]

In some instances, L¹ may comprise an amide moiety. For example, in some instances, L¹ may comprise:

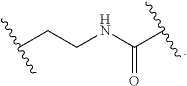 [SMILES: *CCNC(*)=O]

In various instances, the recurring unit of formula (I) may be repeated 3 times to 50 times. In some instances, the recurring unit of formula (I) may be repeated 3 times to 35 times; 5 times to 45 times; 10 times to 40 times; 15 times to 35 times; 20 times to 30 times; or 22 times to 28 times. In some instances, the recurring unit of formula (I) may be repeated no greater than 50 times; no greater than 45 times; no greater than 40 times; no greater than 35 times; no greater than 30 times; no greater than 25 times; no greater than 20 times; no greater than 15 times; no greater than 10 times; or no greater than 5 times. In some instances, the recurring unit of formula (I) may be repeated no less than 3 times; no less than 5 times; no less than 10 times; no less than 15 times; no less than 20 times; no less than 25 times; no less than 30 times; no less than 35 times; no less than 40 times; or no less than 45 times.

The number of repeats of the recurring units of formula (I) can also be expressed as a subscript “n” associated with the recurring unit as typically done in the art with polymers. For example, formula (I) can be denoted as follows

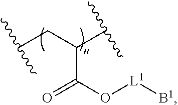

wherein n can be as described above, e.g., 3 to 50. The recurring units of formula (I) can be repeated randomly throughout the polymer, in series, or a combination thereof.

### 2. Recurring Units of Formula (II)

In various instances, the polymer may further comprise recurring units of formula (II):

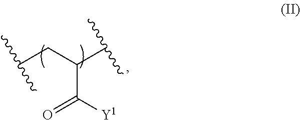

wherein: Y¹ is —NR^{x}R^{y} or —OR^{x}; and R^{x} and R^{y} are each independently hydrogen, C₁₋₄ alkyl, or C₃₋₄ cycloalkyl. In some instances, the recurring units of formula (II) may be acrylamide units of formula (II-a):

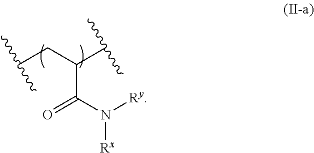

In some instances, R^{x} and R^{y} may each be methyl. Recurring units of formula (II-a) may also be referred to herein as acrylamide units.

In various instances, the recurring unit of formula (II) may be repeated 20 times to 100 times. In some instances, the recurring unit of formula (II) may be repeated 25 times to 95 times; 30 times to 90 times; 35 times to 85 times; 35 times to 70 times; 40 times to 80 times; 45 times to 75 times; 50 times to 70 times; or 55 times to 65 times. In some instances, the recurring unit of formula (II) may be repeated no greater than 100 times; no greater than 90 times; no greater than 80 times; no greater than 70 times; no greater than 60 times; no greater than 50 times; no greater than 40 times; or no greater than 30 times. In some instances, the recurring unit of formula (II) may be repeated no less than 20 times; no less than 30 times; no less than 40 times; no less than 50 times; no less than 60 times; no less than 70 times; no less than 80 times; or no less than 90 times.

The number of repeats of the recurring units of formula (II) can also be expressed as a subscript “m” associated with the recurring unit as typically done in the art with polymers. For example, formula (II) can be denoted as follows

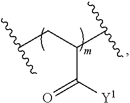

wherein m can be as described above, e.g., 20 to 100. The recurring units of formula (II) can be repeated randomly throughout the polymer, in series, or a combination thereof.

In various instances, the molar ratio of the units of formula (II) to the units of formula (I) may be about 1:1 to about 20:1. In some instances, the molar ratio of the units of formula (II) to the units of formula (I) may be about 1:1 to about 19:1; about 2:1 to about 18:1; about 3:1 to about 17:1; about 4:1 to about 16:1; about 5:1 to about 15:1; about 6:1 to about 14:1; about 7:1 to about 13:1; about 8:1 to about 12:1; or about 9:1 to about 11:1. In some instances, the molar ratio of the units of formula (II) to the units of formula (I) may be no greater than about 20:1; no greater than about 18:1; no greater than about 15:1; no greater than about 12:1; no greater than about 10:1; no greater than about 8:1; no greater than about 5:1; or no greater than about 2:1. In some instances, the molar ratio of the units of formula (II) to the units of formula (I) may be no less than about 1:1; no less than about 2:1; no less than about 5:1; no less than about 8:1; no less than about 10:1; no less than about 12:1; no less than about 15:1; or no less than about 18:1. For example, in some instances, the molar ratio of the units of formula (II) to the units of formula (I) may be about 5:1.

### 3. Molecular Weight

The term “molecular weight” in relation to the polymer refers to number average molecular weight (Mₙ) unless noted otherwise. Molecular weight can be measured by standard techniques known within the art, such as gel permeation chromatography, size exclusion chromatography, and/or rheological analysis. In some instances, the polymer's Mₙ may be measured by gel permeation chromatography.

In various instances, the polymer may have a Mₙ of about 3,000 g/mol to about 30,000 g/mol, as measured by gel permeation chromatography. In some instances, the polymer may have a Mₙ of about 5,000 g/mol to about 25,000 g/mol; about 7,000 g/mol to about 23,000 g/mol; about 10,000 g/mol to about 20,000 g/mol; about 12,000 g/mol to about 18,000 g/mol; or about 14,000 g/mol to about 16,000 g/mol, as measured by gel permeation chromatography. In some instances, the polymer may have a Mₙ of no greater than about 30,000 g/mol; no greater than about 25,000 g/mol; no greater than about 20,000 g/mol; no greater than about 15,000 g/mol; no greater than about 10,000 g/mol; or no greater than about 5,000 g/mol, as measured by gel permeation chromatography. In some instances, the polymer may have a Mₙ of no less than about 3,000 g/mol; no less than about 5,000 g/mol; no less than about 10,000 g/mol; no less than about 15,000 g/mol; no less than about 20,000 g/mol; or no less than about 25,000 g/mol, as measured by gel permeation chromatography.

In various instances, the polymer may have a Mₙ of about 5,000 g/mol to about 8,500 g/mol, as measured by gel permeation chromatography. In some instances, the polymer may have a Mₙ of about 5,500 g/mol to about 8,500 g/mol; about 6,000 g/mol to about 8,000 g/mol; about 6,500 g/mol to about 7,500 g/mol; about 5,500 g/mol to about 6,500 g/mol; or about 7,000 g/mol to about 7,500 g/mol. In some instances, the polymer may have a Mₙ of no greater than about 8,500 g/mol; no greater than about 8,000 g/mol; no greater than about 7,500 g/mol; no greater than about 7,000 g/mol; no greater than about 6,500 g/mol; or no greater than about 6,000 g/mol. In some instances, the polymer may have a Mₙ of no less than about 5,500 g/mol; no less than about 6,000 g/mol; no less than about 6,500 g/mol; no less than about 7,000 g/mol; no less than about 7,500 g/mol; or no less than about 8,000 g/mol.

### 4. General Synthesis

Polymers comprising B¹, e.g., a polymer comprising recurring units of formula (I) and formula (II), may be synthesized by polymerization techniques known within the art. For example, in various embodiments, the disclosed polymer can be synthesized via reversible addition-fragmentation chain-transfer (RAFT) polymerization using monomers that provide recurring units of formula (I) and formula (II). Example monomers that can provide recurring units of formula (I) include

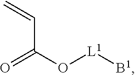

wherein L¹ and B¹ are defined herein. In embodiments where B¹ is not included in the monomer, recurring units of formula (I) can be provided by post-synthetically modifying said recurring unit as shown in General Scheme 1 and General Scheme 2. In addition, an example monomer that can provide recurring units of formula (II) includes

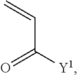

wherein Y¹ is defined herein.

In the following general schemes B¹ are as defined herein. General schemes 1 and 2 below, show an example synthesis for preparing a polymer comprising recurring units of formula (I) where L¹

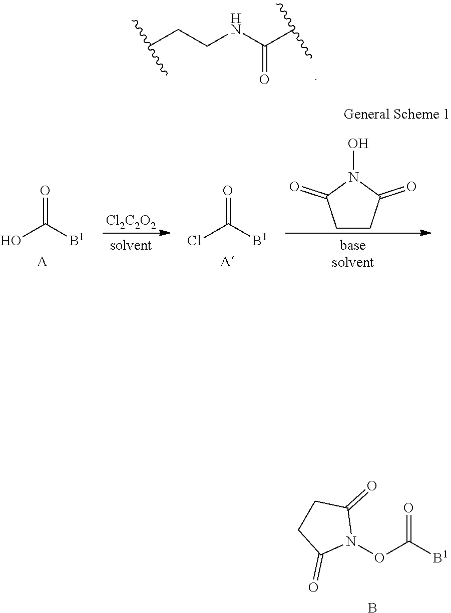

As shown in General Scheme 1, B¹-substituted carboxylic acids of formula A may be reacted with oxalyl chloride under suitable reaction conditions to provide acyl chloride intermediates of formula A′. Then acyl chloride intermediate A′ may then be reacted with N-Hydroxysuccinimide (NHS) under suitable conditions, e.g., in presence of a base such as triethyl amine to form B¹-substituted NHS-esters of formula B.

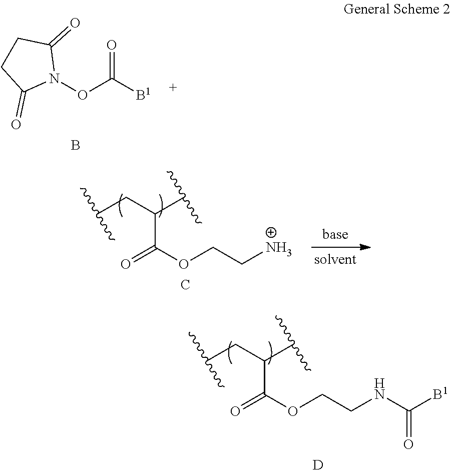

As shown in General Scheme 2, B¹-substituted NHS-esters of formula B may be reacted with a polymer comprising recurring units of formula C under suitable conditions, such as in presence of a base (e.g., triethylamine) to prepare a polymer comprising recurring units of formula D.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, aerotactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### B. Hydrogels

In various instances, the polymer comprising B¹ may be included in a hydrogel. As used herein, the term “hydrogel” is a three-dimensional polymeric structure that is insoluble in water or other aqueous media, but which is capable of absorbing and retaining water. In various instances, the hydrogel may comprise the polymer crosslinked with a diol crosslinker. In some instances, insulin, an insulin variant, an insulin analogue, glucagon, GLP-1, or a combination thereof may be encapsulated within the hydrogel. Exemplary insulin variants or analogues include but are not limited to Humolog® (insulin lispro); Novolog® (insulin aspart); Lantus®, Toujeo®, Basaglar®, Semglee®, (insulin glargine); Levemir® (insulin detemir); Apidra® (insulin glulisine), among others. In some instances, insulin, an insulin variant, an insulin analogue, glucagon, GLP-1, or a combination thereof may be encapsulated within the hydrogel.

### 1. Diol Crosslinker

In various instances, the diol crosslinker may be any diol-based molecule (e.g., polymer) having at least 2 diol moieties that is capable of crosslinking the polymer comprising B¹, e.g., to form a covalent linkage. In some instances, the diol crosslinker comprises a plurality of diol moieties. In some instances, the diol crosslinker comprises at least 2 diol moieties, at least 3 diol moieties, at least 4 diol moieties, at least 5 diol moieties, at least 6 diol moieties, at least 7 diol moieties, at least 8 diol moieties, at least 9 diol moieties, or at least 10 diol moieties. In some instances, the covalent linkage is a dynamic covalent linkage. As used herein the term “dynamic covalent bond” refers to a covalent bond that can reversibly form and dissociate. For example, the constitution of dynamic systems can respond to changes in chemical environment (e.g., complexing entities) or physical conditions (e.g., temperature, mechanical stress, electric field, irradiation).

Suitable diol crosslinkers for crosslinking the polymer may include various polymer diols, such as, polyvinyl alcohol, natural polyols (e.g., tannic acid), macromolecular polyols, and combinations thereof. In some instances, the diol crosslinker comprises a multi-armed polymer, wherein each individual arm comprises a diol of formula (III):

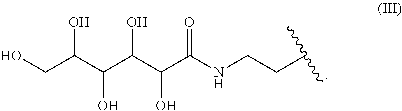 [SMILES: *CCNC(=O)C(O)C(O)C(O)C(O)CO]

For example, in some instances, the diol crosslinker comprises a four-armed polymer, wherein each individual arm comprises a diol of formula (III).

In some instances, the multi-armed polymer comprises a polyalkylene glycol. In various instances, the diol crosslinker comprises:

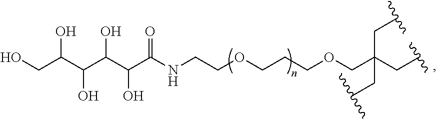

wherein n is 2 to 250.

### 2. Methods for Preparing Hydrogel

In various instances, the hydrogels described herein may be prepared by combining a polymer comprising B¹ with a diol crosslinker in solution to form a crosslinking reaction mixture. In various instances, the crosslinking mixture may be mixed to provide the hydrogel comprising the polymer crosslinked with the diol crosslinker.

In various instances, the crosslinking reaction mixture may be mixed for a time period of 1 to 100 sec.

In various instances, the crosslinking reaction mixture may be mixed at a temperature of 1° C. to 45° C.

In some instances, before mixing the crosslinking reaction mixture, the polymer comprising B¹ and the diol crosslinker may each be dissolved in separate solutions, then combined.

In various instances, before mixing the crosslinking reaction mixture, the pH of the reaction mixture may be adjusted to a pH of 6.5 to 8.5.

In some instances, the hydrogel may be prepared by crosslinking the polymer comprising B¹ with the diol crosslinker at a particular molar ratio of B¹ of the polymer to the diol of the diol crosslinker. The molar ratio of B¹ of the polymer to the diol of the diol crosslinker may be determined by methods known within the art. In some instances, the molar ratio of B¹ of the polymer to the diol of the diol crosslinker may be determined by NMR and/or FTIR. Then, the ratio would be adjusted by mixing ratio of the two polymers.

In various instances, the molar ratio of B¹ of the polymer to the diol of the diol crosslinker may be from about 0.25:1 to about 10:1. In some instances, the molar ratio of B¹ of the polymer to the diol of the diol crosslinker may be from about 0.5:1 to about 10:1; about 0.75:1 to about 9:1; about 1:1 to about 9:1; about 1.5:1 to about 8.5:1; about 2:1 to about 8:1; about 2.5:1 to about 7.5:1; about 3:1 to about 7:1; about 3.5:1 to about 6.5:1; or about 4:1 to about 6:1. In some instances, the molar ratio of B¹ of the polymer to the diol of the diol crosslinker may be no greater than 10:1; no greater than 9:1; no greater than 8:1; no greater than 7:1; no greater than 6:1; no greater than 5:1; no greater than 4:1; no greater than 3:1; no greater than 2:1; no greater than 1:1; or no greater than 0.5:1. In some instances, the molar ratio of B¹ of the polymer to the diol of the diol crosslinker may be no less than 0.25:1; no less than 0.5:1; no less than 0.75:1; no less than 1:1; no less than 2:1; no less than 3:1; no less than 4:1; no less than 5:1; no less than 6:1; no less than 7:1; no less than 8:1; or no less than 9:1.

### C. Pharmaceutical Compositions

In various instances, insulin encapsulated within a hydrogel (i.e., “hydrogel-encapsulated insulin”) may be included in a pharmaceutical composition. Insulin, insulin, insulin variants, insulin analogues, glucagon, GLP-1, or combination thereof can also be similarly included in a pharmaceutical composition. Hydrogel-encapsulated insulin may be incorporated into pharmaceutical compositions suitable for administration to a subject (such as a patient, which may be a human or non-human). The pharmaceutical compositions may include a “therapeutically effective amount” or a “prophylactically effective amount” of the active agent (insulin or analogues or variants thereof). A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A “therapeutically effective amount” is also one in which any toxic or detrimental effects are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable excipients. The term “pharmaceutically acceptable excipient,” as used herein, means a non-toxic, inert solid, semi-solid or liquid filler, diluent, encapsulating material, or formulation auxiliary of any type. Some examples of materials which can serve as pharmaceutically acceptable excipients are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The disclosed compositions can be topically administered. Topical compositions, such as a topical composition comprising a disclosed hydrogel and a pharmaceutically acceptable excipient, may be applied locally to the skin. The pharmaceutically acceptable excipient of the topical composition may aid penetration of the hydrogels into the skin. The pharmaceutically acceptable excipient may further include one or more optional components.

The amount of the pharmaceutically acceptable excipient employed in conjunction with the hydrogel-encapsulated insulin is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} ed., (1976).

A pharmaceutically acceptable excipient may include a single ingredient or a combination of two or more ingredients. For example, in topical compositions, the pharmaceutically acceptable excipient includes a topical excipient. Suitable topical excipients include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, excipients for skin applications include propylene glycol, dimethyl isosorbide, and water, and even more particularly, phosphate buffered saline, isotonic water, deionized water, monofunctional alcohols, and symmetrical alcohols.

The pharmaceutically acceptable excipient of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%. The amount of thickener(s) in a topical composition is typically about 0% to about 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%. The amount of fragrance in a topical composition is typically about 0% to about 0.5%, particularly, about 0.001% to about 0.1%. Suitable pH adjusting additives include HCl or NaOH in amounts sufficient to adjust the pH of a topical pharmaceutical composition.

### Devices

Further disclosed herein are devices comprising a microneedle array. The microneedle array can comprise a plurality of microneedles on a surface of a substrate. Each microneedle can include the polymer disclosed herein crosslinked with the diol crosslinker disclosed herein. Accordingly, the description above for the polymer and the diol crosslinker can also be applied to the disclosed devices.

In various instances, the diol crosslinker can be a multi-armed polymer, wherein each individual arm comprises a diol of formula (III):

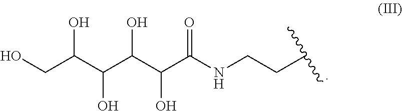 [SMILES: *CCNC(=O)C(O)C(O)C(O)C(O)CO]

In some instances, the diol crosslinker comprises:

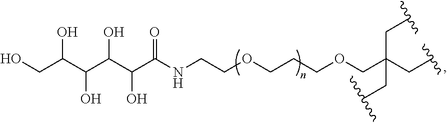

wherein n is 2 to 250.

In various instances, the molar ratio of the units of formula (II) to the units of formula (I) is about 1:1 to about 20:1 for the polymer of the microneedles. In some instances, the molar ratio of the units of formula (II) to the units of formula (I) is about 5:1 for the polymer of the microneedles.

In various instances, the molar ratio of B¹ of the polymer to the diol of the diol crosslinker is about 0.25:1 to about 10:1 for the polymer crosslinked with the diol of the microneedles. In some instances, the molar ratio of B¹ of the polymer to the diol of the diol crosslinker is about 4:1 to about 8:1 for the polymer crosslinked with the diol of the microneedles.

The microneedle array can be prepared in any suitable shape. Example shapes include a square shape, a rectangular shape, a circular shape, an oval shape, and a letter shape. In addition, the shape of the microneedle is not limited. Examples of microneedle shapes include a conical shape, a circular truncated cone shape, a quadrangular pyramid shape, a triangular pyramid shape, and a konide-like shape. In addition, the microneedle array can include different populations of microneedles where the different populations have different shapes. The number of microneedles in the array is also generally not limited. Example arrays include 2×2, 4×4, 5×5, 10× 10, 10×20, 20×20, 50×10, 50×50, 100×100, and the like.

In various instances, the microneedle array can have a surface area of about 50 mm² to about 2000 mm².

In various instances, each microneedle has a length of about 300 μm to about 800 μm. In some instances, each microneedle has a length of about 400 μm to about 700 μm; about 500 μm to about 600 μm; about 300 μm to about 500 μm; or about 500 μm to about 800 μm. In some instances, each microneedle has a length of no greater than 800 μm; no greater than 700 μm; no greater than 600 μm; or no greater than 500 μm. In some instances, each microneedle has a length of no less than 300 μm; no less than 400 μm; no less than 500 μm; or no less than 600 μm. Length of the microneedles can be measured by techniques known within the art, such as scanning electron microscopy.

In some instances, each microneedle is solid. In some instances, each microneedle lacks a channel extending through the length of the microneedle. In some instances, each microneedle has a channel extending through the length of the microneedle. In some instances, the plurality of microneedles can be a combination of the foregoing. For example, an individual microneedle, in some instances, can be solid, lack a channel extending through the length of the microneedle, or have a channel extending through the length of the microneedle.

In various instances, the substrate can comprise the same polymer crosslinked with the diol crosslinker as each microneedle. In other words, in some instances, the substrate can be made from the same materials as the plurality of microneedles.

In various instances, each microneedle can have a failure point of greater than 0.05 N/needle, greater than 0.1 N/needle, greater than 0.2 N/needle, greater than 0.3 N/needle, greater than 0.4 N/needle, greater than 0.5 N/needle, greater than 0.6 N/needle, greater than 0.7 N/needle, or greater than 0.8 N/needle. In some instances, each microneedle can have a failure point of greater than 0.6 N/needle. The failure point can be measured by techniques known within the art, such as by dynamic mechanical analysis using, e.g., a rheometer.

In various instances, each microneedle can comprise insulin, insulin variants, insulin analogues, glucagon, GLP-1, or combinations thereof. In some instances, each microneedle can comprise insulin. In some instances, at least a portion of the plurality of microneedles can comprise insulin, an insulin variant, an insulin analogue, glucagon, or a combination thereof. In some instances, at least a portion of the plurality of microneedles can comprise insulin.

### A. Methods for Making the Devices

Also disclosed are methods for making the devices comprising a microneedle array. The method can comprise adding the disclosed hydrogel to a mold, the mold comprising a plurality of microneedle molds. The number, shape, and dimensions of the molds is generally not limited and can correspond to the description of the microneedles and array thereof described above. In addition, the mold can include populations of different shapes and populations of different dimensions (e.g., lengths). And, because the disclosed methods of making the devices include the hydrogel as disclosed herein, the description above for the hydrogel, the polymer, and the diol crosslinker can be applied to the methods of making the devices.

The method can further comprise applying a force to the hydrogel such that the hydrogel fills each microneedle mold. Examples of applying force to the hydrogel include vacuum and centrifugation. In some instances, the mold is centrifuged, thereby applying a force to the hydrogel such that the hydrogel fills each microneedle mold. Application of the force to the hydrogel can aid in drying the hydrogel.

The method can also include drying the hydrogel to provide a device comprising a microneedle array, the microneedle array comprising a plurality of microneedles that align in number and arrangement with the plurality of microneedle molds. In some instances, drying the hydrogel is done by placing in a desiccator. Drying the hydrogel provides a plurality of microneedles, wherein each microneedle compromises the dehydrated hydrogel.

In various instances, insulin, insulin variants, insulin analogues, glucagon, or GLP-1, or combinations thereof are encapsulated in the hydrogel.

In various instances, the method does not include crosslinking the hydrogel during or after adding to the mold. In other words, the method can circumvent the toxicity concerns of in situ polymerization associated with most microneedle fabrication methods.

### Methods

Disclosed herein are methods for delivering insulin to a subject in need thereof. The method can comprise contacting an area of the subject's skin with the device as disclosed herein including a therapeutic such as insulin. Upon contacting the area of the subject's skin, the microneedle array of the device can pierce the skin of the subject, thereby transdermally delivering the insulin to the subject. In some instances, the subject has diabetes. In instances where the subject has diabetes, the method can also be used to treat the subject. In such instances, the amount of insulin can be a therapeutically effective amount as described herein. The method can also be applied to delivering insulin, insulin variants, insulin analogues, glucagon, or GLP-1, or combinations thereof.

The description of the microneedle array and device thereof above can be applied to the methods of delivering insulin. In addition, when applicable, pharmaceutical compositions as described above can be used in providing the disclosed devices, and thus can be used in the methods of delivering insulin, insulin variants, insulin analogues, glucagon, or GLP-1, or combinations thereof.

One embodiment described herein is a polymer comprising:
(i) recurring units of formula (I):

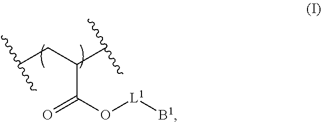

wherein:
L¹ is a linking moiety; and
B¹ is

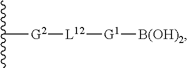

wherein:
G¹ is a pyridylene or a phenylene, wherein G¹ is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₃₋₄ cycloalkyl, —NO₂, or —OC₁₋₂ haloalkyl;
L¹² is a C₁₋₆ alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and
G² is phenylene, wherein G² is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₁₋₂ haloalkyl, —OC₃₋₄ cycloalkyl, or —NO₂; and
(ii) recurring units of formula (II)

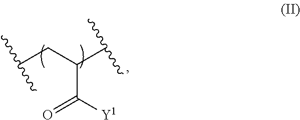

wherein:
Y¹ is —NR^{x}R^{y} or —OR^{x}; and
R^{x} and R^{y} are each independently hydrogen, C₁₋₄ alkyl, or C₃₋₄ cycloalkyl.
   In one aspect, B¹ comprises:

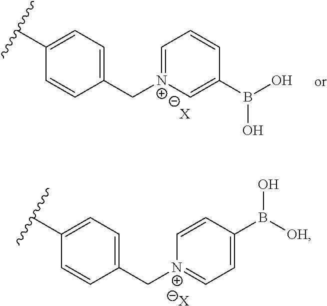

wherein: X^{Θ} is an anion having a net charge of −1. In another aspect, X^{Θ} is Br “, Cl⁻, NO₃”, H₂PO₄, H₂PO₃⁻, HSO₄, HSO₃⁻, H₃C—SO₃⁻, HCO₃⁻, HCO₂, H₃C—CO₂⁻, HC₂O₄⁻, or TsO⁻. In another aspect, X^{Θ} is Br⁻ or Cl⁻. In another aspect, B¹ comprises:

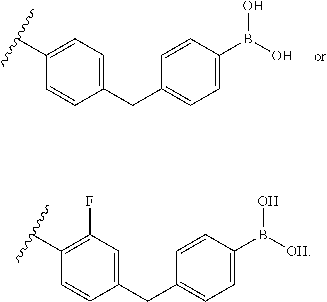 [SMILES: *c1ccc(Cc2ccc(B(O)O)cc2)cc1 ; *c1ccc(Cc2ccc(B(O)O)cc2)cc1F]

In another aspect, L¹ comprises an amide moiety.

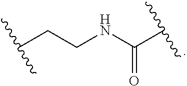 [SMILES: *CCNC(*)=O]

In another aspect, L¹ comprises
In another aspect, the recurring units of formula (II) are acrylamide units of formula (II-a):

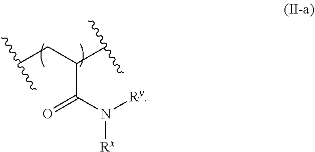

In another aspect, R^{x} and R^{y} are each methyl. In another aspect, the molar ratio of the units of formula (II) to the units of formula (I) is about 1:1 to about 20:1. In another aspect, the molar ratio of the units of formula (II) to the units of formula (I) is about 5:1. In another aspect, the polymer has a number average molecular weight (Mₙ) of about 3,000 g/mol to about 30,000 g/mol as measured by gel permeation chromatography. In another aspect, the polymer has a Mₙ of about 5,000 g/mol to about 8,500 g/mol as measured by gel permeation chromatography. In another aspect, the recurring unit of formula (I) is repeated 3 times to 50 times. In another aspect, the recurring unit of formula (II) is repeated 20 times to 100 times.

Another embodiment described herein is a hydrogel comprising a polymer described herein crosslinked with a diol crosslinker. In one aspect, the molar ratio of B¹ of the polymer to the diol of the diol crosslinker is from about 0.25:1 to about 10:1. In another aspect, insulin, an insulin variant, an insulin analogue, glucagon, GLP-1, or a combination thereof is encapsulated within the hydrogel.

Another embodiment described herein is a hydrogel comprising: a polymer described herein crosslinked with a diol crosslinker including a multi-armed polymer, wherein each individual arm comprises a diol of formula (III):

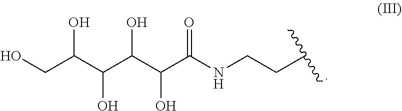 [SMILES: *CCNC(=O)C(O)C(O)C(O)C(O)CO]

In one aspect, the molar ratio of B¹ of the polymer to the diol of the diol crosslinker is from about 0.25:1 to about 10:1. In another aspect, the multi-armed polymer comprises a polyalkylene glycol. In another aspect, the multi-armed polymer is a four-armed or an eight-armed polymer.

Another embodiment described herein is a pharmaceutical composition comprising insulin, an insulin variant, an insulin analogue, glucagon, or GLP-1, or a combination thereof encapsulated within a hydrogel described herein, and a pharmaceutically acceptable excipient.

Another embodiment described herein is a device comprising: a microneedle array comprising a plurality of microneedles on a surface of a substrate, each microneedle comprising a polymer described herein crosslinked with a diol crosslinker, the diol crosslinker including: a multi-armed polymer, wherein each individual arm comprises a diol of formula (III):

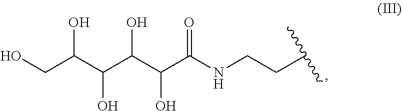 [SMILES: *CCNC(=O)C(O)C(O)C(O)C(O)CO]

wherein the molar ratio of the units of formula (II) to the units of formula (I) is about 1:1 to about 20:1, and the molar ratio of B¹ of the polymer to the diol of the diol crosslinker is about 0.25:1 to about 10:1. In one aspect, the diol crosslinker comprises:

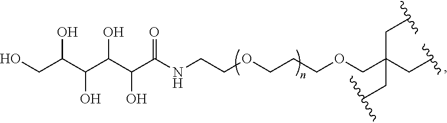

wherein n is 2 to 250. In another aspect, the molar ratio of the units of formula (II) to the units of formula (I) is about 5:1. In another aspect, the molar ratio of B¹ of the polymer to the diol of the diol crosslinker is about 4:1 to about 8:1. In another aspect, each microneedle has a length of about 300 μm to about 800 μm. In another aspect, each microneedle lacks a channel extending through the length of the microneedle. In another aspect, the substrate comprises the same polymer crosslinked with the diol crosslinker as each microneedle. In another aspect, each microneedle has a failure point of greater than 0.6 N/needle. In another aspect, each microneedle further comprises insulin, an insulin variant, an insulin analogue, glucagon, GLP-1, or a combination thereof.

Another embodiment described herein is a method of making a device comprising a microneedle array, the method comprising: adding a hydrogel described herein to a mold, the mold comprising a plurality of microneedle molds; applying a force to the hydrogel such that the hydrogel fills each microneedle mold; and drying the hydrogel to provide a device comprising a microneedle array, the microneedle array comprising a plurality of microneedles that align in number and arrangement with the plurality of microneedle molds, wherein each microneedle comprises the dehydrated hydrogel. In one aspect, the insulin, insulin variant, insulin analogue, glucagon, or GLP-1, or combination thereof is encapsulated in the hydrogel. In another aspect, the method does not include crosslinking of the hydrogel during or after adding to the mold.

Another embodiment described herein is a method of delivering insulin to a subject in need thereof, the method comprising: contacting an area of the subject's skin with the device of claim 24, wherein the insulin, insulin variant, insulin analogue, glucagon, GLP-1, or combination thereof is transdermally delivered to the subject. In one aspect, the subject has diabetes.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A polymer comprising:
   (i) recurring units of formula (I):

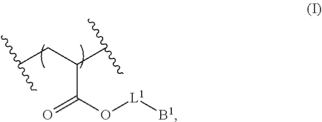

wherein:
L¹ is a linking moiety; and
B¹ is

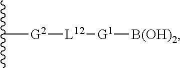

wherein:
G¹ is a pyridylene or a phenylene, wherein G¹ is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₃₋₄ cycloalkyl, —NO₂, or —OC₁₋₂ haloalkyl;
L¹² is a C₁₋₆ alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and
G² is phenylene, wherein G² is optionally substituted with 1-2 substituents independently selected m halogen, —CN, C₁₋₄ alkyl, C₃₋₄ cycloalkyl, C₁₋₂ haloalkyl, —OC₁₋₄ alkyl, —OC₁₋₂ haloalkyl, OC₃₋₄ cycloalkyl, or —NO₂; and
(ii) recurring units of formula (II)

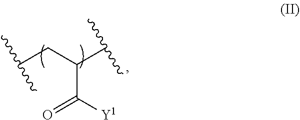

wherein:
   Y¹ is —NR^{x}R^{y} or —OR^{x}; and
   R^{x} and R^{y} are each independently hydrogen, C₁₋₄ alkyl, or C₃₋₄ cycloalkyl.
Clause 2. The polymer of clause 1, wherein B¹ comprises:

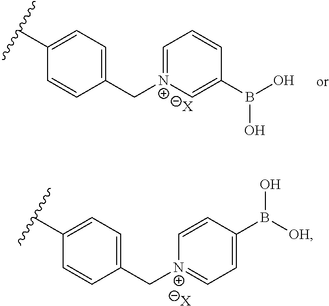

wherein:
X^{Θ} is an anion having a net charge of −1.
Clause 3. The polymer of clause 2, wherein X^{Θ} is Br⁻, Cl⁻, NO₃⁻, H₂PO₄⁻, H₂PO₃, HSO₄, HSO₃⁻, H₃C—SO₃⁻, HCO₃⁻, HCO₂⁻, H₃C—CO₂⁻, HC₂O₄⁻, or TsO⁻.
Clause 4. The polymer of clause 2, wherein X^{Θ} is Br⁻ or Cl⁻.
Clause 5. The polymer of any one of clauses 1-4, wherein B¹ comprises:

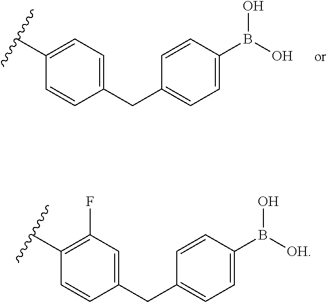 [SMILES: *c1ccc(Cc2ccc(B(O)O)cc2)cc1 ; *c1ccc(Cc2ccc(B(O)O)cc2)cc1F]

Clause 6. The polymer of any one of clauses 1-5, wherein L¹ comprises an amide moiety.
Clause 7. The polymer of any one of clauses 1-6, wherein L¹ comprises

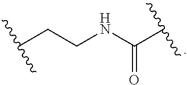 [SMILES: *CCNC(*)=O]

Clause 8. The polymer of any one of clauses 1-7, wherein the recurring units of formula (II) are acrylamide units of formula (II-a):

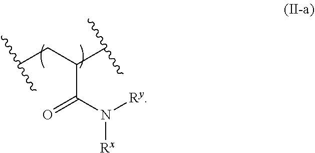

Clause 9. The polymer of any one of clauses 1-8, wherein R^{x} and R^{y} are each methyl.
Clause 10. The polymer of any one of clauses 1-9, wherein the molar ratio of the units of formula (II) to the units of formula (I) is about 1:1 to about 20:1.
Clause 11. The polymer of any one of clauses 1-10, wherein the molar ratio of the units of formula (II) to the units of formula (I) is about 5:1.
Clause 12. The polymer of any one of clauses 1-11, wherein the polymer has a number average molecular weight (Mₙ) of about 3,000 g/mol to about 30,000 g/mol as measured by gel permeation chromatography.
Clause 13. The polymer of any one of clauses 1-12, wherein the polymer has a Mₙ of about 5,000 g/mol to about 8,500 g/mol as measured by gel permeation chromatography.
Clause 14. The polymer of any one of clauses 1-13, wherein the recurring unit of formula (I) is repeated 3 times to 50 times.
Clause 15. The polymer of any one of clauses 1-14, wherein the recurring unit of formula (II) is repeated 20 times to 100 times.
Clause 16. A hydrogel comprising the polymer of any one of clauses 1-15 crosslinked with a diol crosslinker.
Clause 17. The hydrogel of clause 16, wherein the molar ratio of B¹ of the polymer to the diol of the diol crosslinker is from about 0.25:1 to about 10:1.
Clause 18. The hydrogel of clause 16 or 17, wherein insulin, an insulin variant, an insulin analogue, glucagon, GLP-1, or a combination thereof is encapsulated within the hydrogel.
Clause 19. A hydrogel comprising:
   the polymer of any one of clauses 1-18, crosslinked with a diol crosslinker including a multi-armed polymer, wherein each individual arm comprises a diol of formula (III):

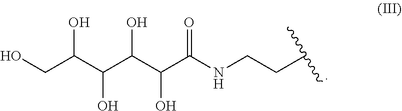 [SMILES: *CCNC(=O)C(O)C(O)C(O)C(O)CO]

Clause 20. The hydrogel of any one of clauses 19 or 20, wherein the molar ratio of B¹ of the polymer to the diol of the diol crosslinker is from about 0.25:1 to about 10:1.
Clause 21. The hydrogel of any one of clauses 19-21, wherein the multi-armed polymer comprises a polyalkylene glycol.
Clause 22. The hydrogel of clause 21, wherein the multi-armed polymer is a four-armed or an eight-armed polymer.
Clause 23. A pharmaceutical composition comprising insulin, an insulin variant, an insulin analogue, glucagon, or GLP-1, or a combination thereof encapsulated within the hydrogel of any one of clauses 19-22, and a pharmaceutically acceptable excipient.
Clause 24. A device comprising:
   a microneedle array comprising a plurality of microneedles on a surface of a substrate, each microneedle comprising the polymer of any one of clauses 1-15 crosslinked with a diol crosslinker, the diol crosslinker including:
   a multi-armed polymer, wherein each individual arm comprises a diol of formula (III):

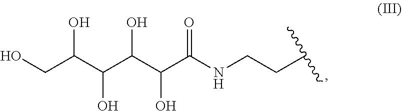 [SMILES: *CCNC(=O)C(O)C(O)C(O)C(O)CO]

wherein the molar ratio of the units of formula (II) to the units of formula (I) is about 1:1 to about 20:1, and the molar ratio of B¹ of the polymer to the diol of the diol crosslinker is about 0.25:1 to about 10:1.
Clause 25. The device of clause 24, wherein the diol crosslinker comprises:

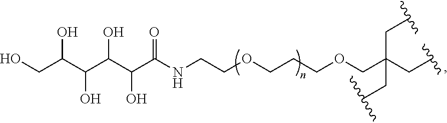

wherein n is 2 to 250.
Clause 26. The device of clause 24 or 25, wherein the molar ratio of the units of formula (II) to the units of formula (I) is about 5:1.
Clause 27. The device of any one of clauses 24-26, wherein the molar ratio of B¹ of the polymer to the diol of the diol crosslinker is about 4:1 to about 8:1.
Clause 28. The device of any one of clauses 24-27, wherein each microneedle has a length of about 300 μm to about 800 μm.
Clause 29. The device of any one of clauses 24-28, wherein each microneedle lacks a channel extending through the length of the microneedle.
Clause 30. The device of any one of clauses 24-29, wherein the substrate comprises the same polymer crosslinked with the diol crosslinker as each microneedle.
Clause 31. The device of any one of clauses 24-30, wherein each microneedle has a failure point of greater than 0.6 N/needle.
Clause 32. The device of any one of clauses 24-31, wherein each microneedle further comprises insulin, an insulin variant, an insulin analogue, glucagon, GLP-1, or a combination thereof.
Clause 33. A method of making a device comprising a microneedle array, the method comprising:
   adding the hydrogel of any one of clauses 19-22 to a mold, the mold comprising a plurality of microneedle molds;
   applying a force to the hydrogel such that the hydrogel fills each microneedle mold; and
   drying the hydrogel to provide a device comprising a microneedle array, the microneedle array comprising a plurality of microneedles that align in number and arrangement with the plurality of microneedle molds, wherein each microneedle comprises the dehydrated hydrogel.
Clause 34. The method of clause 33, wherein the insulin, insulin variant, insulin analogue, glucagon, or GLP-1, or combination thereof is encapsulated in the hydrogel.
Clause 35. The method of clause 33, wherein the method does not include crosslinking of the hydrogel during or after adding to the mold.
Clause 36. A method of delivering insulin to a subject in need thereof, the method comprising: contacting an area of the subject's skin with the device of any one of clauses 24⁻, wherein the insulin, insulin variant, insulin analogue, glucagon, GLP-1, or combination thereof is transdermally delivered to the subject.
Clause 37. The method of clause 36, wherein the subject has diabetes.

### EXAMPLES

### Material and Polymer Synthesis

Synthetic procedures for the preparation of monomers, polymers, and macromers are shown in Scheme 1.

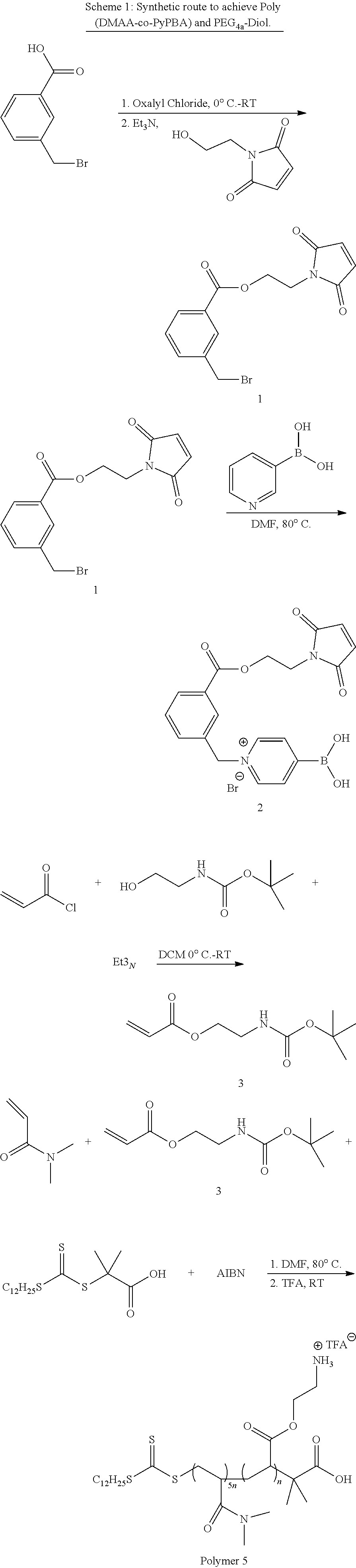

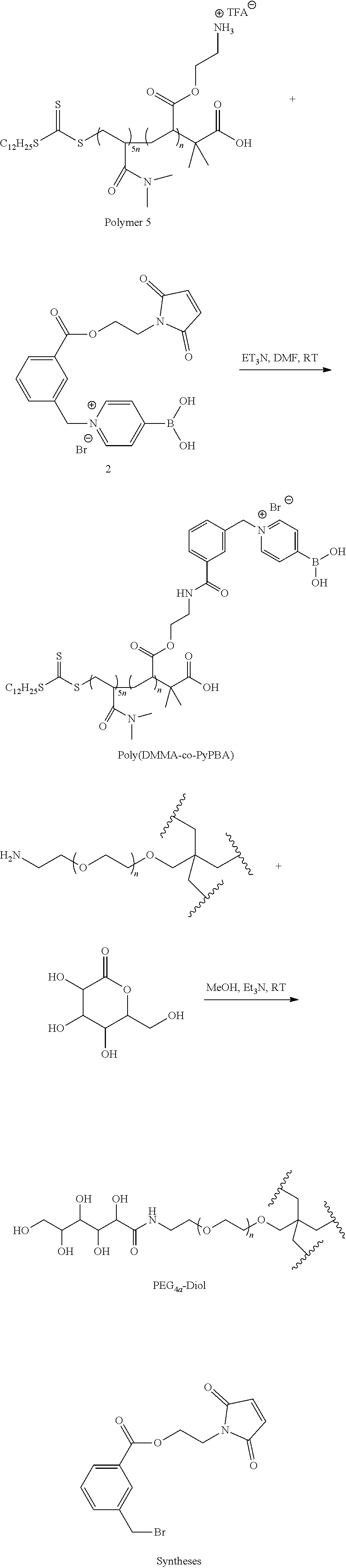

### Preparation of 3-(bromomethyl)benzoic Acid-NHS (1)

To a 50 mL oven-dried round bottom flask, 3-(bromomethyl)benzoic acid (1 g, 4.6 mmol) was charged and diluted with a solvent mixture of dry THF (4 mL), DCM (8 mL), and DMF (20 μL) and the mixture was stirred in an ice bath for 15 min before treatment with oxalyl chloride (1.9 mL, 23 mmol). After addition, the mixture was kept on ice for another 5 min and then stirred at ambient temperature for 90 min. Solvent was removed under vacuum at 50° C. and the residue was diluted with DCM (20 mL). The mixture was then added dropwise to a DCM (20 mL) solution of N-Hydroxysuccinimide (0.63 g, 5.5 mmol) and triethylamine (0.8 mL, 5.5 mmol) at 0° C. and the mixture was then warmed to ambient temperature for further reaction. After 16 h, the reaction was quenched with 40 mL 1 N HCl (aq) and the mixture was transferred to a separation funnel. The organic layer was separated and washed with water (40 mL) and sat. NaCl (40 mL) and dried over Na₂SO₄. The mixture was then filtered and concentrated to yield white solids and used directly for the next step without purification. The spectrum pure product could be obtained by column chromatography eluting with hexane and ethyl acetate as white solids with the yield of 80% by mole. ¹H NMR (400 MHZ, Chloroform-d) δ 8.16 (t, J=1.9 Hz, 1H), 8.09 (ddd, J=9.3, 7.2, 1.5 Hz, 1H), 7.72 (dt, J=7.7, 1.6 Hz, 1H), 7.53 (q, J=7.8 Hz, 1H), 4.63 (s, 1H), 4.52 (s, 1H), 2.90 (d, J=15.9 Hz, 4H).

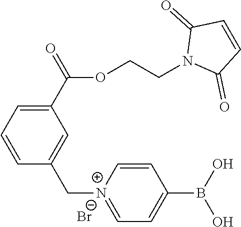 [SMILES: O=C(OCCN1C(=O)C=CC1=O)c1cccc(C[n+]2ccc(B(O)O)cc2)c1 ; [Br-]]

### Preparation of PyPBA-NHS (2)

To a 100 mL oven-dried round bottom flask compound 1 (1.4 g, 4.6 mmol) and pyridin-3-ylboronic acid (0.56 g, 4.6 mmol) were charged and diluted with 25 mL dry DMF. The mixture was then stirred at 70° C. for 24 h before being cooled down to ambient temperature. The solvent was concentrated to a small volume and then treated with 90 mL THF. The undissolved solids were collected and washed with THF, DCM, and Et₂O as the final product with yield of 76% by mole. ¹H NMR (400 MHZ, D₂O) δ 8.79 (s, 1H), 8.71 (s, 1H), 8.66 (d, J=7.3 Hz, 2H), 8.59 (t, J=5.7 Hz, 2H), 8.20 (s, 1H), 7.89 (t, J=6.6 Hz, 2H), 7.81 (d, J=7.6 Hz, 1H), 7.66 (t, J=8.1 Hz, 1H), 5.83 (s, 2H), 2.98 (s, 4H).

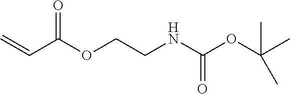

### Preparation of 2-(^{t}Boc-amino)ethyl Acrylate (3)

To a 100 mL oven-dried round bottom flask, tert-butyl (2-hydroxyethyl) carbamate (3 g, 18.6 mmol) and triethylamine (4 mL, 28 mmol) were added and diluted with 20 mL DCM. The mixture was stirred at 0° C. for 15 min before the addition of acryloyl chloride (2.3 mL, 28 mmol). After addition, the mixture was stirred at 0° C. for another 15 min before being warmed to ambient temperature. The reaction was quenched after 24 h by adding 40 mL water and the mixture was transferred to a separation funnel. The organic layer was separated and washed with saturated NaCl (aq) and dried over Na₂SO₄. The mixture was then filtered and concentrated and the residue was loaded on the column eluting with hexane and ethyl acetate for purification. The final product was collected as a white solid with a yield of 70% by mole. ¹H NMR (400 MHZ, Chloroform-d) δ 6.43 (dd, J=17.3, 1.4 Hz, 1H), 6.13 (dd, J=17.4, 10.5 Hz, 1H), 5.86 (dd, J=10.5, 1.4 Hz, 1H), 4.79 (s, 1H), 4.22 (t, J=5.3 Hz, 2H), 3.44 (q, J=5.6 Hz, 2H), 1.45 (s, 9H).

### Copolymerization of N,N′-Dimethylacrylamide and 2-(tert-Butoxycarbonylamino)ethyl Acrylate (4)

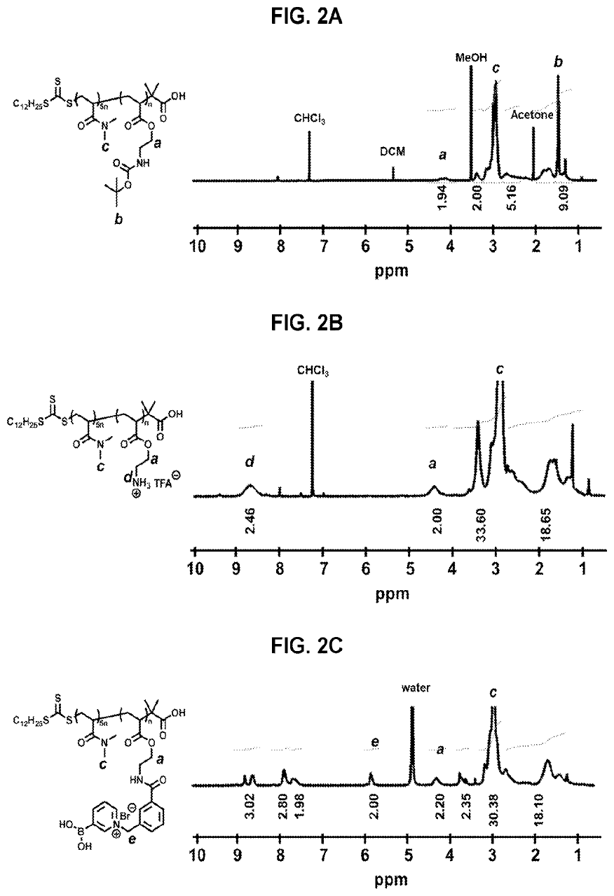

A mixture of N,N′-Dimethylacrylamide (DMAA, 2 g, 20 mmol), Compound 3 (0.86 g, 4 mmol), 2-(((dodecylthio) carbonothioyl)thio)-2-methylpropanoic acid (87 mg, 0.24 mmol), and AIBN (5.2 mg, 0.032 mmol) were added to a 10 mL oven-dried round bottom flask and diluted with 5 mL DMF. The mixture was degassed for 2 h and then stirred at 80° C. After 4 h, the mixture was exposed to the air and then concentrated under vacuum. The residue was diluted with 10 mL MeOH before being transferred to a dialysis tube (3,500 MWCO) and dialyzed against a MeOH-Acetone-DCM (1:1:1) mixture for 4 h followed by precipitation in cold ether. The undissolved solids were recovered as the target product with a mass recovery of 2.6 g. ¹H NMR (400 MHZ, Chloroform-d, FIG. 2A) õ 4.11 (d, J=41.0 Hz, 2H), 3.42-3.21 (m, 2H), 3.21-2.78 (m, 31H), 2.62 (s, 5H), 1.95-1.13 (m, 22H).

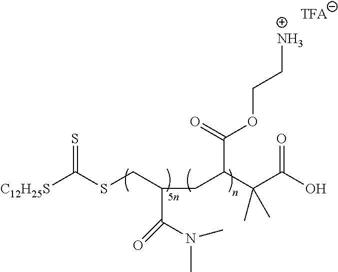

### T-Boc Deprotection (5)

In a 50 mL oven-dried round bottom flask Polymer 4 (3 g) was treated with 20 mL TFA. The mixture was stirred at ambient temperature for 2 h and then concentrated under vacuum. The residue was diluted with MeOH, and the target product was precipitated as a white solid into cold ether with a mass recovery of 1.5 g. ¹H NMR (400 MHZ, Chloroform-d, FIG. 2B) õ 8.69 (s, 2H), 4.45 (s, 2H), 4.00-2.52 (m, 33H), 2.49-1.04 (m, 18H).

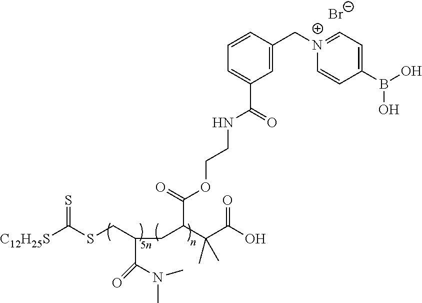

### Preparation of Poly(DMAA-Co-PyPBA)

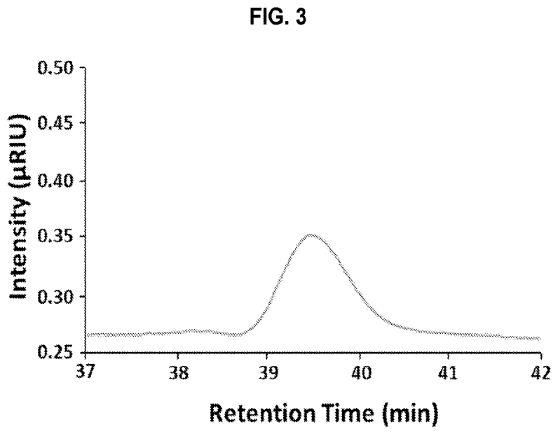

To a 100 mL oven-dried round bottom flask, Polymer 5 (20 mmol) and triethylamine (1.4 mL, 10 mmol) were added and diluted with 50 mL dry DMF. The mixture was stirred at ambient temperature for 15 min before addition of Compound 2 (2.15 g, 5 mmol). After 24 h, the solvent was removed, and the residue was diluted with 10 mL MeOH before being transferred to a dialysis tube (3,500 MWCO) and dialyzed against MeOH for 24 h. Then the mixture was concentrated to a small volume and precipitated into the cold ether. The undissolved solids were collected as the final product with a mass recovery of 2.1 g. The molecular weight of the polymer was determined by GPC as MW=6471 g/mol (PDI=1.01) (FIG. 3). ¹H NMR (400 MHZ, D₂O, FIG. 2C) õ 8.64 (d, J=74.6 Hz, 3H), 7.81 (s, 3H), 7.60 (s, 2H), 5.76 (s, 2H), 4.22 (s, 2H), 3.77-3.45 (m, 2H), 2.88 (s, 31H), 2.58-1.09 (m, 18H).

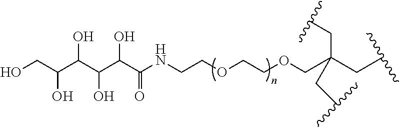

### Preparation of PEG₄ₐ-Diol

The preparation of PEG₄ₐ-Diol was performed as described by Yesilyurt et al., Adv. Mat. 28 (1): 86-91 (2016). Briefly, PEG₄ₐ-NH₂ (10 kDa, 3.0 g, 0.3 mmol, Laysan Bio, Inc), D-gluconolactone (0.3 g, 4.8 mmol), and triethylamine (0.7 mL, 4.8 mmol) were added to a 100 ml oven-dried round-bottom flask and diluted with 50 mL MeOH. The mixture was stirred for 3 d at ambient temperature before being transferred to a dialysis tube (3,500 MWCO) and dialyzed against MeOH for 24 h, followed by further dialysis against DI water for 24 h. The product was collected and lyophilized. ¹H NMR (400 MHZ, D₂O) δ 4.29 (d, J=3.7 Hz, 1H), 4.05 (t, J=3.2 Hz, 1H), 3.67 (s, 193H), 3.51-3.38 (m, 5H).

### Gel Permeation Chromatography

Polymer average molecular weight was characterized by gel permeation chromatography (GPC) using a Thermo Scientific Ultimate 3000 HPLC system (Dionex), a RI refractometer (ERC, RefractoMax520), and a polymer-based GPC column (Shodex™, OHpak SB-804 HQ) operating at room temperature. The system was calibrated with a series narrow-distributed PEO standards (Agilent Technologies, 545000, 272400, 117900, 48290, 21230, 16100, 8160, 3860, 1450 and 610 g/mol). A sample at 0.1 mg/mL was injected in the GPC system, while a water (w/0.1 M NaN₃) mobile phase was running at a stable 0.2 mL/min flow rate for 60 min.

### Hydrogel Formation

Hydrogels were prepared at 5 wt %, 7.5 wt %, or 10 wt % by first dissolving Poly(DMAA-co-PyPBA) and PEG₄ₐ-Diol separately at the desired concentrations (5 wt %, 7.5 wt %, or 10 wt %). The dissolved solutions were adjusted to neutral pH using small volumes of 0.1 M NaOH or HCl as needed and then mixed at a molar ratio of 4:1 of the PyPBA to Diol motifs for gelation.

### Dynamic Oscillatory Rheology

Hydrogels at 5 wt %, 7.5 wt %, or 10 wt % were prepared as described above by suspending polymers in PBS containing 0, 100, 200, and 400 mg/dl glucose to mimic physiological glucose levels. Dynamic oscillatory rheology was performed using a HR-2 Discovery Hybrid Rheometer (TA Instruments) with a 25 mm parallel plate geometry. The gap was set at 200 μm. An amplitude sweep (10 rad/s, 0.1-2000% strain) was first performed to ensure that a subsequent frequency sweep (1% strain, 0.1-200 rad/s) was conducted in the linear viscoelastic regime. Shear viscosity measurements were performed on 10 wt % hydrogels with a shear rate ramping from 0.1 s⁻¹ to 50 s⁻¹. Step-strain measurements were performed on 10 wt % hydrogels at 10 rad/s cycling strain between 1% and 1000%.

### Fabrication of Microneedles

Polydimethylsiloxane (PDMS) microneedle molds were purchased as a custom-made product from Blueacre Technology Ltd. and used as the template for fabrication. The microneedle molds were in a 20×20 array with mold dimensions yielding a conical shape with a base diameter of 300 μm, a height of 600 μm, and a tip-to-tip spacing of 600 μm. The resulting 20×20 arrays therefore had an area of roughly 144 mm² (12 mm×12 mm). For certain studies, this patch was cut into sections in order to enable more studies to be performed on a single device. A premixed 10 wt % hydrogel (1 mL) prepared in DI water was loaded onto the PDMS mold and restrained on the top of the mold by a custom-cut PDMS holder sized to hold the array and restrain the material from flowing off of the array. The loaded hydrogel was centrifuged at 4255×g and 37° C. for 3 h to fill the cavities of the mold, remove air bubbles, and dry the gel solution within the mold. After further drying in a desiccator overnight, the microneedle patch was peeled from the PDMS mold.

### Scanning Electron Microscopy

The microneedle patch prepared as described above was sputter coated with a 5 nm Pd/Au using an EM ACE600 sputter coater (Leica). The morphology of the microneedles was imaged using an environmental scanning electron microscope (ESEM, Thermo Scientific Prisma) at an accelerating voltage of 10 kV.

### Fluorescence Microscopy

A 10 wt % hydrogel containing 0.1 wt % FITC-labeled insulin was prepared in DI water and used to fabricate a microneedle patch, as described. The microneedles were imaged using an EVOS FL Auto fluorescence microscope (Life Technologies) at 4× with illumination from a GFP light cube.

### Compression Testing

The mechanical properties of the microneedle patch prepared as described above were assessed by dynamic mechanical analysis (DMA) using a HR-2 Discovery Hybrid Rheometer. A patch of 9×9 array was attached to the bottom stainless-steel flat plate. The top stainless-steel flat plate was lowered vertically at a constant speed of 0.01 mm/s. The displacement and axial force were recorded until the maximum axial force limit of 50 N.

### In Vitro Release of FITC-Insulin

Using the cap of a 3 mL glass vial as a mold, 200 μL of 5 wt % hydrogel with 0.025 wt % FITC-insulin was dried to form a film in the cap under conditions resembling those used to form dried microneedles. Each vial cap was glued to one well of a 12-well plate and submerged in 11 mL of PBS or PBS with 400 mg/dl glucose (n=5/group). At each timepoint, 20 μL of the solution was drawn into a black 96-well plate and replaced by 20 μL fresh PBS or PBS with 400 mg/dl glucose. The collected solution was diluted with 180 μL of PBS or PBS with 400 mg/dL glucose. Fluorescence was measured using a Tecan Infinite M200 PRO microplate reader (Ex: 485 nm, Em: 520 nm). A standard curve of FITC-insulin in the range of 1 ng/ml to 20 μg/mL was used to calculate the cumulative release of FITC-insulin. For swelling and erosion studies, the material was prepared as a dried film by these same methods and added to pre-weighed vials. The initial total mass (vial+sample) was recorded, and the samples were then incubated in PBS without or with the addition of 400 mg/dl glucose. Excess liquid was removed at subsequent times and the residual vial and same were weighed, with the mass ratio determined from the mass at the given time, Mₜ, and the initial mass M₀.

### Diabetic Rat Study

A streptozotocin (STZ)-induced diabetic rat model was established to assess the ability of the microneedle patch in preventing hyperglycemia. The studies were detailed in a protocol approved by the University of Notre Dame Animal Care and Use Committee and adhered to all relevant Institutional, State, and Federal guidelines. Male Sprague Dawley rats (200-230 g) were fasted for 8 h and then administered a single intraperitoneal (i.p.) injection of 10 mg/mL STZ at a dose of 65 mg/kg. Rats were provided with water containing 10% sucrose for 24 h following STZ injection. Diabetes was verified 7 d following STZ treatment using a hand-held blood glucose meter (CVS brand) by tail-vein blood collection to ensure blood glucose level (BGL) following a 12 h fast was >250 mg/dL. Each microneedle patch (10×10 array) was loaded with 5 IU insulin (calculated on the basis of needle volume only) and prepared from 10 wt % hydrogels by first filter-sterilizing the precursor solutions and then fabricating microneedles under aseptic conditions by the methods noted above. Microneedles without insulin were used as a control group (n=4/group) and otherwise prepared identically. The dorsal skin of rats was shaved and then animals were subsequently fasted for 12 h. Following fasting, the initial BGL was measured. The microneedle patch was pressed onto the rat skin and covered by a Tegaderm Transparent Film Dressing (3M Corporation). BGLs were subsequently monitored for 12 h.

### Histology

One microneedle patch (10×10 array) prepared as described in the in vivo studies was pressed on the rat skin for 5 s and then removed from the skin. The rat was euthanized, and the surrounding tissue was harvested, fixed in formalin for 48 h, stored in 70% ethanol, and subjected to histological processing, sectioning, and staining with H & E. Bright-field images were taken using an EVOS FL Auto microscope at 10×.

### Polymer Design

To prepare microneedles from dynamic-covalent crosslinked polymer networks, a synthetic polymeric precursor (Scheme 1) was first synthesized by RAFT polymerization, targeting a 5:1 monomer ratio of N,N′-Dimethylacrylamide (DMAA) and 2-(^{t}Boc-amino)ethyl acrylate (^{Boc}NEA) (FIG. 2A). The peak at 1.4 ppm (peak b) was assigned to methyl protons of the tBoc group, with an integral of 9. Peaks from 3.75-4.25 ppm, which integrate to 30, are the signature peaks of methyl protons on the main polymer backbone. Accordingly, the ^{Boc}NEA block consisted of ˜17% of the synthesized polymer, effectively matching the target of 16.67% established by the monomer feed ratio. The DMAA was selected as the majority component due to the excellent solubility of polyDMAA in water and the high mechanical strength of the polymer arising from the self-hydrogen bonding capacity DMAA side chains. Mechanical properties were an important consideration when designing microneedles to be prepared using dynamic-covalent crosslinking so as to ensure these had the requisite rigidity and strength to penetrate the dermal layer. The introduction of ^{Boc}NEA provides a protected amine sidechain on the polymer for subsequent post-synthetic modification, thereby dictating the crosslink density of the resulting network. The monomer ratio is thus a parameter that may be tunable in future iterations of this approach toward modifying glucose-sensitivity and insulin release kinetics, though it is expected that DMAA would remain the majority component for consideration of the mechanical properties necessary for needle formation and dermal penetration. Upon tBoc deprotection, the primary amine was revealed (FIG. 2B), enabling sites for further modification with PBA-containing groups via amide bond formation. This deprotection proceeded to completion, evidenced by elimination of the ^{t}Boc protons (formerly at 1.4 ppm) and emergence of a broad signal (peak d) at 8.75 ppm corresponding to protons of the primary amine.

PBA-diol chemistry is widely explored and used in preparing dynamic-covalent networks due to the high affinity of binding for PBA to glucose-like diols provided from appending a glucano-δ-lactone (GdL) moiety to a polymeric or macromeric building block (FIG. 1). However, the high-affinity binding of most PBA groups to this diol chemistry may limit glucose-responsive function of the bond; this is in addition to challenges arising from susceptibility of many PBA-diol bonds to interference from non-glucose analytes. A previously reported pyridinium boronic acid (PyPBA) group was shown to afford glucose binding (K_{eq}=166.7 M⁻¹) that was almost 20-fold higher than was achieved using traditional PBA chemistry (K_{eq}=8.6 M⁻¹); the improved glucose-binding affinity of PyPBA was also accompanied by a ˜2-fold reduction in binding to common competing analytes such as fructose and lactate compared to a traditional PBA. Critical to this observation is the low pKₐ of PyPBA (pKₐ˜4.4) arising from the adjacent pyridinium salt, making this a stronger acid than traditional PBAs. Accordingly, PyPBA was selected here as the glucose-binding motif for use in preparing glucose-responsive microneedles from PBA-diol crosslinking.

For post-synthetic modification of the deprotected poly(DMAA-co-NEA), an N-hydroxysuccinimide (NHS) activated PyPBA (PyPBA-NHS) was synthesized for polymer modification. Briefly, acylation of 3-(bromomethyl)benzoic acid with oxalyl chloride was followed by incorporation of NHS, after which the intermediate was reacted with pyridin-3-ylboronic acid to yield the amine-reactive PyPBA-NHS at 76% yield. Modification of poly(DMAA-co-NEA) proceeded quantitatively, yielding the desired poly(DMAA-co-PyPBA) with a molecular weight determined from GPC (FIG. 3) of MW=6471 g/mol (PDI=1.01), suggesting the degree of polymerization is 70. The ¹H NMR spectrum of the final product (FIG. 2C) revealed PyPBA-specific signatures, with the methylene protons adjacent to the pyridinium salt (peak e) at 5.75 ppm integrating to 2 and protons in the aromatic region (7.25-9.00 ppm) integrating to 8. Relative to a reference methylene on the NEA group (peak a), these values supported complete modification of available amines with the PyPBA motif.

### Glucose-Responsive Polymer Hydrogel Networks

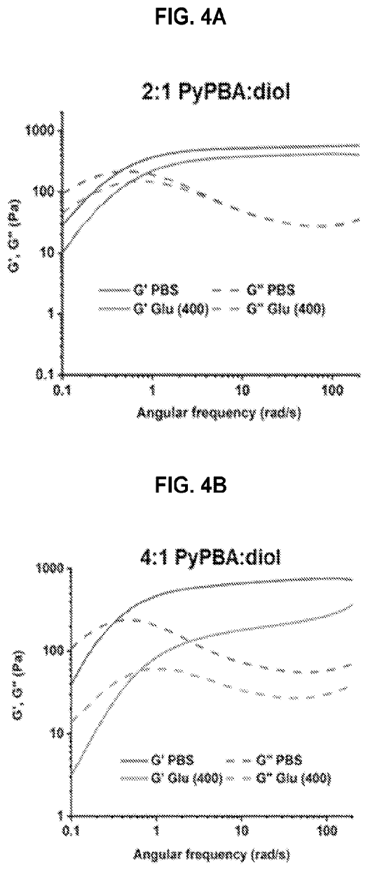

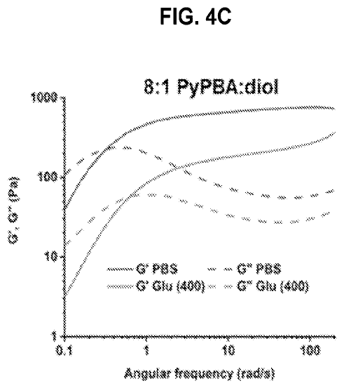

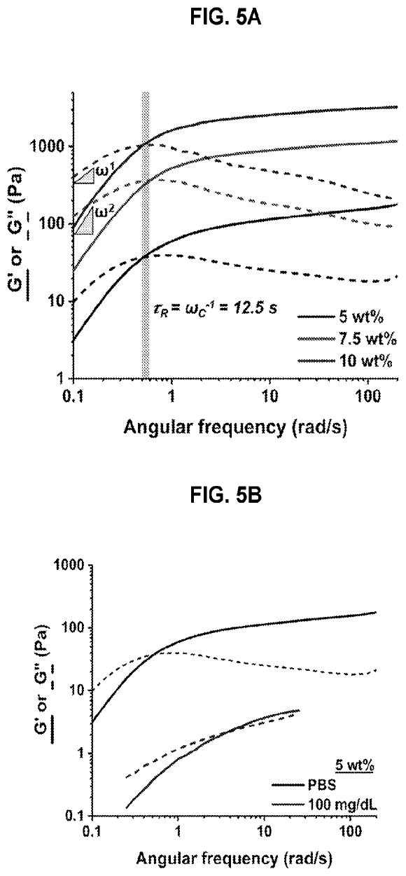

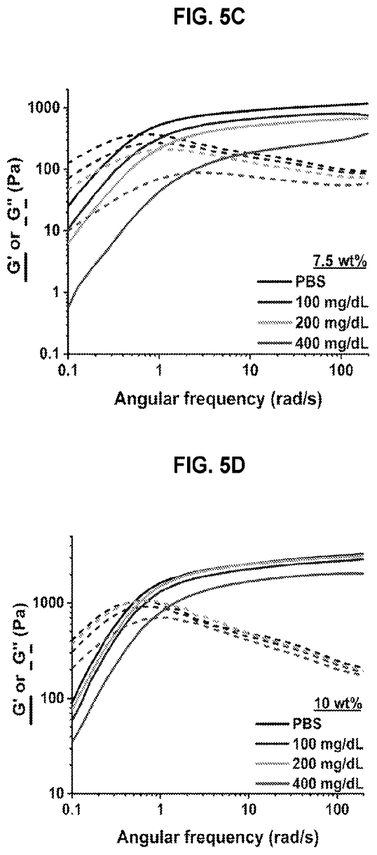

Though eventual microneedle arrays would cast hydrogels in molds prior to drying, it was expected that these would readily rehydrate by imbuing interstitial fluid of the transcutaneous space to reform hydrogel-like character within the skin. Accordingly, a study of the glucose-responsive properties of these dynamic-covalent polymer networks in their hydrogel state was next performed. To prepare hydrogels, a diol-bearing macromer (PEG₄ₐ-Diol) was synthesized as previously described and mixed with poly(DMAA-co-PyPBA) at a ratio of excess PyPBA groups (FIG. 2D). See Yesilyurt et al., Adv. Mat. 28(1): 86-91 (2016). Four PyPBA motifs for each diol moiety on the 4-arm PEG macromer was used for the work shown here. This ratio was motivated by preliminary rheological studies confirming that an excess of PyPBA improved glucose responsiveness of the hydrogel, with ratios of PBA:diol of 4:1 and 8:1 having a greater change in mechanical properties than 2:1 upon exposure to glucose (FIG. 4). The rate of crosslink exchange in dynamic-covalent networks, and the influence of ambient glucose level on PBA-diol bond formation and dynamics, is especially useful to understanding the function of these materials. Oscillatory frequency sweeps were thus collected at a constant strain value of 1%, determined to be within the linear viscoelastic regime, in order to investigate the dynamic viscoelastic behaviors of networks prepared from PyPBA-diol dynamic-covalent crosslinks (FIG. 5).

The polymer/macromer mixtures at the fixed ratio of four PyPBA motifs per one diol were first prepared in water at concentrations of 5%, 7.5%, and 10% (w/v) and analyzed by a frequency sweep. The critical frequency (ωc) where G′=G″ is related to both the network relaxation time (T_{R}) and dissociation rate of bonding (K_{off}=T_{R}⁻¹) in dynamically associating network. This is because under oscillatory deformation, energy remaining in bonds over the timescale of oscillation is accounted for in G′, while bonds that reorganize at a rate in excess of this time constant of oscillation account for energy loss (G″); thus, the G′/G″ crossover is correlated with the time constant for average bond lifetime in a network.

The traces for hydrogels prepared at different concentrations had effectively identical dynamic properties, with T_{R} estimated to be ˜12.5 s for the networks at each of the 3 concentrations evaluated (FIG. 5A). This finding supports dynamic-covalent bonding of PyPBA to diol as the dominant contributor to hydrogel mechanics and dynamics. Meanwhile, in the terminal regime assessed at low frequency the behavior of all materials scaled with frequency in a manner consistent with terminal relaxation and linear viscoelasticity (G′≈ω² and G″≈ω′). Accordingly, these networks demonstrated canonical Maxwell behavior, evidenced by both concentration-independent G′/G″ crossover and terminal relaxation. In the plateau regime (ω>>ωc), G′ of the network scales with concentration, as expected due to a higher density of network crosslinking in hydrogels prepared at higher concentration.

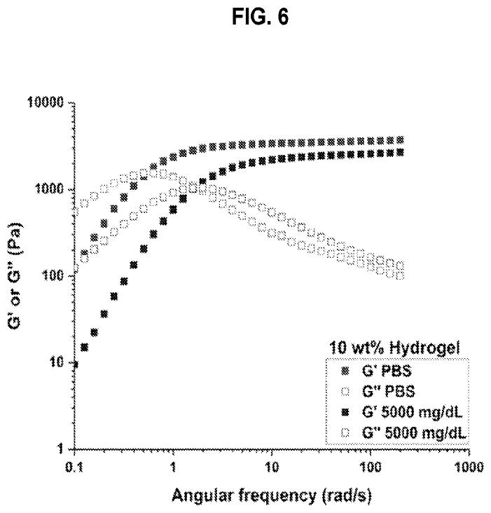

The glucose-responsive crosslinking of these hydrogel networks prepared at each of the three different polymer concentrations (5%, 7.5%, and 10% w/v) was next explored using oscillatory rheology (FIG. 5B-D). As glucose concentration increased across a physiologically relevant range from 0 mg/dL to 400 mg/dL, general trends were observed for materials at all three polymer concentrations, though the magnitude of glucose-responsive function varied considerably. For the 5 wt % hydrogels, mechanical properties were critically altered in the presence of glucose and only hydrogels prepared in 100 mg/dl glucose offered data above the limits of instrument sensitivity (FIG. 5B). These hydrogels effectively behaved as a sol, though a G′/G″ crossover was observed corresponding to a reduction in T_{R} from 12.5 s to ˜1.6 s upon addition of glucose, supporting a much more dynamic network. Meanwhile for the 7.5 wt % hydrogels, the mechanical properties steadily decreased upon additional glucose exposure (FIG. 5C), evident in the modulus of the plateau region at high frequency which indicates a reduction in the equilibrium bound state of the network. For these hydrogels, network dynamics also increased with increasing levels of glucose competition, with T_{R} transitioning from ˜12.5 s (0 mg/dL) to ˜7.9 s (100 mg/dL) to ˜6.3 s (200 mg/dL) to ˜3.2 s (400 mg/dL). Finally, at 10 wt % the hydrogels were significantly less sensitive to glucose (FIG. 5D). Only subtle changes in the plateau modulus were evident, and these hydrogels required glucose levels of 400 mg/dL for any appreciable change in mechanical properties or increased network dynamics (T_{R}=˜7.5). Even at a very high glucose level of 5000 mg/dL, the 10 wt % hydrogels had mechanical properties that were minimally impacted (FIG. 6).

Generally, the trend for a reduction in the equilibrium-bound state of the network and an increase in network dynamics as glucose concentration is increased aligns with expectations for the impact of competition from ambient glucose on PyPBA-diol dynamic-covalent crosslinking. The observed loss of glucose-responsiveness as polymer concentration is increased is also expected based on observations of this behavior in other PBA-diol crosslinked networks. This effect is hypothesized to arise from glucose being a less efficient competitor as the local concentration of PBA-binding diol groups on the polymer is increased. Though these networks use PyPBA chemistry with K_{eq} of glucose binding measured at 166.7 M⁻¹, this motif also binds the GdL-derived diol structure used on the PEG₄ₐ-Diol macromer with a K_{eq} of 5280 M⁻¹. In a 5 wt % hydrogel, the effective concentration of macromer-presented diols was 10.7 mM, increasing to 21.4 mM at 10 wt %. Meanwhile, 400 mg/dL of ambient glucose corresponds to a concentration of 22 mM. As such, the ability for glucose to compete with macromer-presented diol, which itself binds PyPBA considerably better, is reduced at higher polymer concentration.

### Fabrication and Characterizations of Microneedles

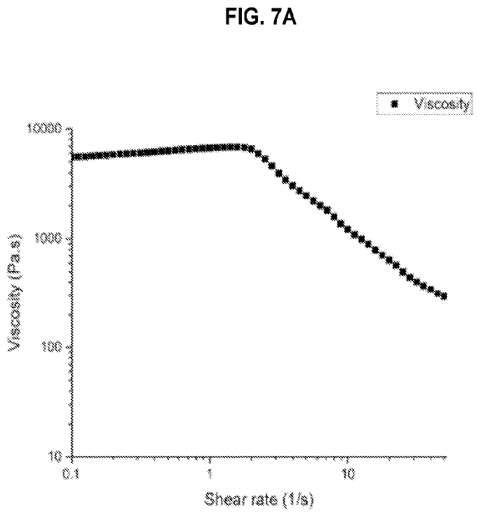

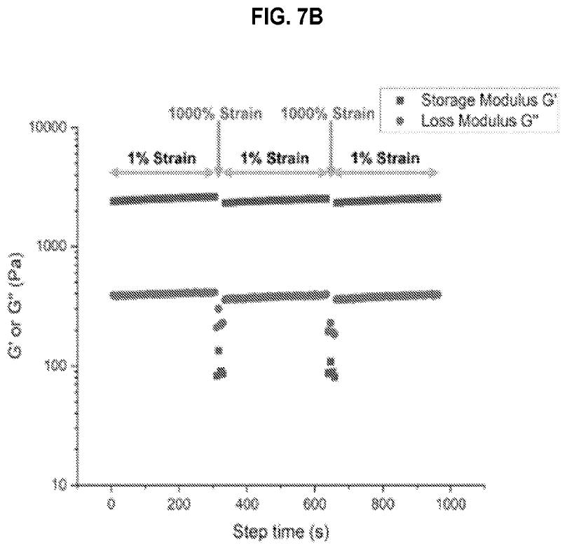

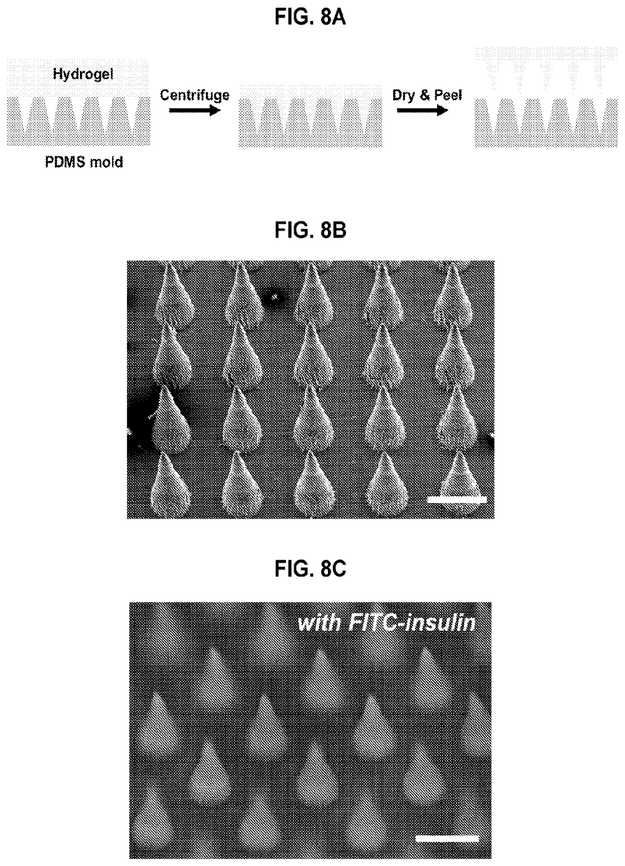

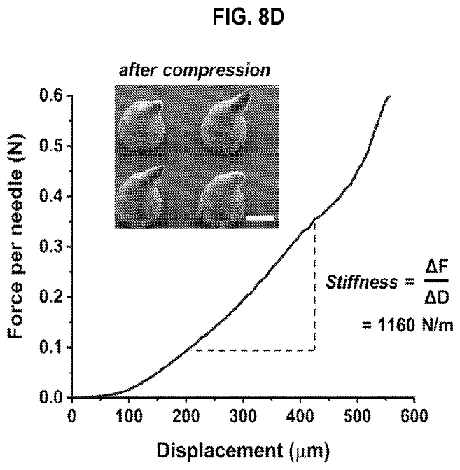

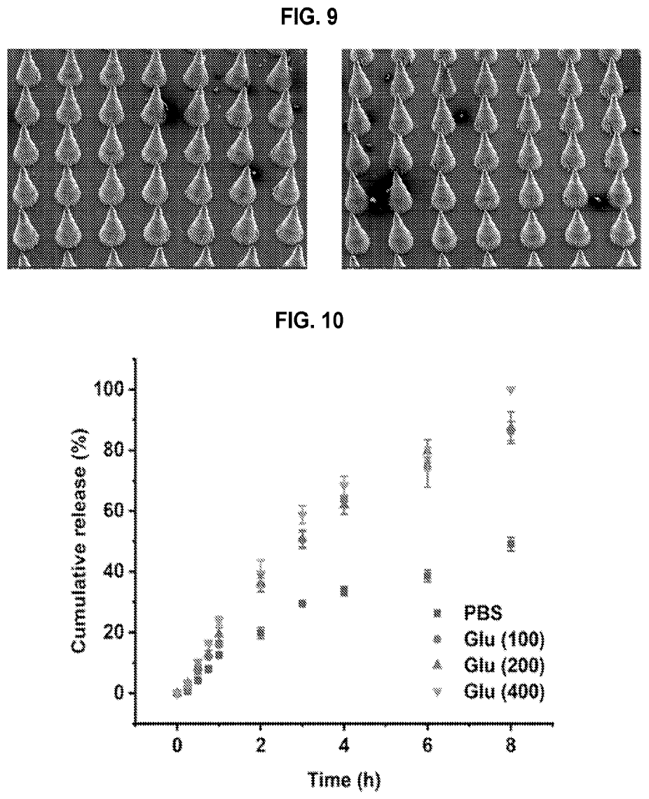

Previous demonstrations of microneedles for insulin delivery have used in situ polymerization with covalent crosslinking performed within filled microneedle molds prior to drying. This route introduces a risk of exposure to toxic unreacted monomers and crosslinking agents as well as (photo) initiators of polymerization. However, loading a microneedle mold with pre-formed covalent polymer hydrogels is not feasible. A benefit of dynamic-covalent hydrogels is their ability to shear-thin and self-heal, allowing these materials to flow under applied mechanical force, fill voids or defects, and recover their original mechanical properties through dynamic bond rearrangement (FIG. 7). Accordingly, preformed hydrogels were explored for their ability to be loaded into microneedle molds with aid of centrifugation (FIG. 8A). The centrifugation was necessary to fill the mold and ensure removal of air bubbles. This step also served as a pre-drying step, with the patch subsequently removed and placed into a desiccator overnight for final drying. After drying, the material could be peeled from the mold, revealing a uniform and intact array of conical microneedles (FIG. 8B). Tip-to-tip spacing (607±8 μm), and base diameter (322±8 μm) were measured at five different places on the SEM micrographs, confirming the array maintains the dimensions of the master mold. Additional microscopy supported the ability of this preparation protocol to yield effectively defect-free arrays (FIG. 9). One drawback to this method of preparing microneedles by drying dynamic materials comes in the challenges of preparing more complex microneedle structures, such as two-layer designs with a drug embedded only in the tip and an inert backing layer, as rehydration and mixing of the tip material and its payload occurs during two-step fabrication. As a model drug, FITC-labeled insulin was loaded along with the premixed hydrogel and processed identically for microneedle fabrication. Fluorescent microscopy revealed a uniform distribution of FITC-labeled insulin in the microneedle patch (FIG. 8C). There was no visual difference in the quality of microneedles produced when insulin was included, attributed to the relatively small amount of insulin in the device, accounting for 1% of total dry weight.

Mechanical strength is an important parameter, ensuring that polymeric microneedles are able to penetrate the stratum corneum for transdermal drug delivery. To assess mechanical strength of these microneedle devices produced from the dying of dynamic-covalent hydrogels using a preformed mold, a 9×9 microneedle array was compressed against a flat stainless-steel plate (FIG. 8D). No failure point was observed up to a maximum force of ˜0.6 N/needle; this value is significantly higher than the required force (˜0.01 N/needle) that has been suggested as necessary to penetrate living human skin. The stiffness can be estimated from the linear region of the force/displacement curve to be ˜1160 N/m. The microneedle array demonstrated some buckling following compression against the stainless-steel plate up to this maximum force, though no signs of a critical fracture event are evident in these deformed needles.

### Insulin Release & Delivery

Networks prepared from PBA-diol dynamic-covalent crosslinks have long been explored as materials for insulin delivery. By this mechanism, glucose (a cis-1,2 diol) competes in a concentration-dependent manner with diols used for material crosslinking, acting to drive release of encapsulated cargo by reducing the extent of network crosslinking, increasing the porosity, and promoting erosion of the material. Accordingly, dynamic-covalent hydrogels were first evaluated for glucose-responsive release of FITC-labeled insulin. In the hydrogel state, the hydrogels demonstrated insulin release that was accelerated in the presence of glucose, though release was not obviously linked to specific glucose concentration (FIG. 10). Though multiple prior studies on 10 wt % PEG-based hydrogels prepared with PBA-diol bonds have shown no glucose-responsive release of insulin, it is difficult to explain the observation here for some glucose-responsive release that was at the same time indifferent to absolute glucose concentration. As the affinity (K_{eq}) reported for PyPBA binding to the diol is roughly 30× that of its binding to glucose, it is conceivable that the bonds in this hydrogel are relatively insensitive to the concentration range of glucose being evaluated, as was the case in these same prior reports for PBA-diol crosslinking of PEG gels. However, in contrast to these prior reports, which had presented equimolar PBA and diol on macromers, the PyPBA group in the present hydrogels is at a 4-fold excess to the diol. Thus, this glucose-induced, yet concentration-independent, increase in insulin release from these hydrogels may arise not from competition for existing PyPBA-diol bonds, but instead through glucose binding to excess PyPBA groups serving to increase the hydrophilicity of the polymer network. As the bulk glucose is in sufficient excess to achieve binding saturation across the full concentration range, one would then expect such a phenomenon to be effectively concentration-independent over the range studied.

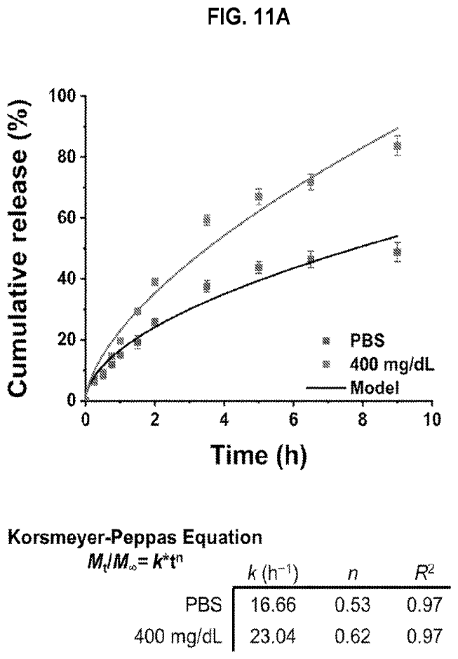

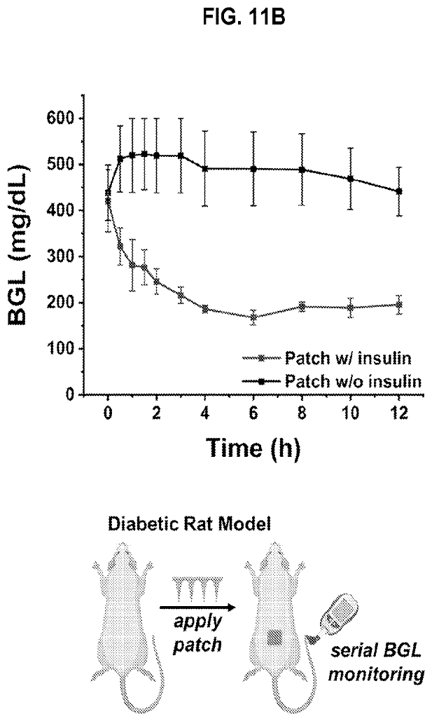

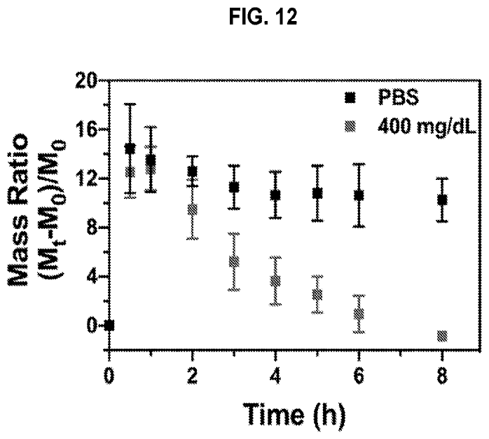

For use in microneedles, however, the network is likely denser as a result of the drying process, with introduction of multi-modal release mechanisms due to the need for rehydration upon initial exposure to the transcutaneous environment. To more closely evaluate this scenario, the release of FITC-labeled insulin was measured from dried films of the material prepared by the same methods and at the same composition as the microneedle arrays. This altered format was required to circumvent mass variations that arise from differential thickness of the backing layer on the microneedle film. Accordingly, the release of FITC-insulin was compared from dried films of the material submerged in PBS versus 400 mg/dL glucose, selected to resemble levels typical of severe hyperglycemia (FIG. 11A). After 9 h, samples exposed to 400 mg/dl had released ˜82% of encapsulated insulin, compared to only ˜48% released in PBS. The rates of release were fit to the Korsmeyer-Peppas equation, revealing a difference in the release rates for samples exposed to 400 mg/dl glucose (22.04%/h) compared to PBS (16.66%/h). Importantly, the exponential term extracted from this model (n) supports a difference in the mechanism of release; release in PBS was consistent with expectations for Fickian diffusion (n˜0.5) while release in 400 mg/dl glucose was indicative of anomalous release to include that caused by swelling and erosion (n>0.5). Studies monitoring the change in mass over time supported glucose-independent swelling and water absorption in these materials upon initial rehydration, with a dramatic increase in subsequent material erosion when glucose was present (FIG. 12), in agreement with the model fit to release studies. Accordingly, these data support network disruption arising from ambient glucose competition with PBA-diol crosslinks in dynamic-covalent networks.

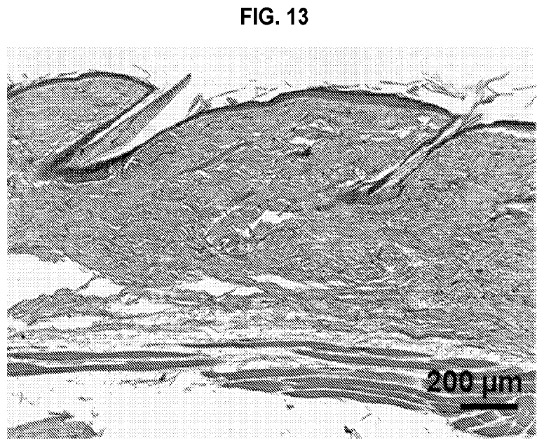

A remaining question concerned whether the microneedles could successfully penetrate the stratum corneum for transdermal delivery of encapsulated insulin in a manner that ensured the therapeutic was bioavailable. As such, in vivo delivery from these dynamic-covalent microneedle patches was assessed for short-term blood glucose control in an STZ-induced diabetic rat model (FIG. 11B). A 12 h fast was performed to normalize blood glucose levels, adjust for variable time since the last meal, and improve insulin sensitivity. Rats that remained hyperglycemic following this fasting were randomly grouped to ensure comparable starting blood glucose levels and administered microneedle patches that were either insulin-free or prepared to contain 25 IU/kg insulin for the entire patch, based on an average rat weight of 225 g. Importantly, only a portion of the incorporated insulin is likely available for delivery, as much of the total mass of the device (and by extension the encapsulated insulin) is in a backing layer that does not penetrate the skin. For treatment with insulin-containing microneedle patches, blood glucose dropped gradually over a period of ˜4 h before normalizing in the range of 180-200 mg/dL for the remaining 8 h of observation; this level is considered a normoglycemic range in healthy rodents. Not surprisingly, rats administered patches that did not contain insulin demonstrated hyperglycemia throughout the duration of the study. This control serves to adjust for changes in blood glucose levels that may arise as a result of the microneedle application procedures or continued fasting. Limitations in the fasting duration for this protocol necessitated an end to the study following 12 h of observation, at which point the treated rats remained on a plane of normoglycemia. Histology on the skin following removal of the path revealed regularly spaced sites where the microneedles had been inserted (FIG. 13). These studies therefore confirmed that the microneedles are able to penetrate the skin to make their encapsulated insulin payload bioavailable, and further that the insulin remains active through the course of device processing. Further studies may seek to expand existing protocols for testing in diabetic rats to explore glucose-responsive function through inclusion of glucose challenges.

Here a polymeric microneedle patch is demonstrated that uses dynamic-covalent PBA-diol bonds in a dual role affording both network crosslinking and glucose sensing. Combining a synthetic polymer post-modified with pendant PBA motifs and a 4-arm PEG macromer terminated with diol moieties resulted in hydrogels with mechanical and dynamic properties dictated by glucose level. These dynamic-covalent hydrogels can be prepared and subsequently loaded into microneedle molds using centrifugation, resulting in a dried patch of uniform microneedles with the requisite mechanical properties to penetrate skin. The release of insulin from dried patches of this material was accelerated in the presence of glucose. Finally, the utility of these materials for the encapsulation and in vivo delivery of insulin was verified in a diabetic rat model. Accordingly, this approach using dynamic-covalent crosslinking facilitates the fabrication of microneedle arrays that circumvent the toxicity concerns of in situ polymerization methods, offering a route toward devices for blood glucose control that are minimally invasive and have a convenient form-factor for delivery.

What is claimed is:

1. A polymer comprising:
(i) recurring units of formula (I): 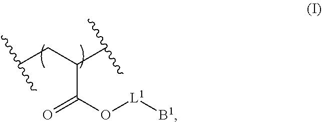
wherein:
L¹ is a linking moiety; and
B¹ is 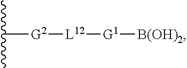
wherein:
G¹ is a pyridylene or a phenylene, wherein G¹ is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₃₋₄ cycloalkyl, —NO₂, or —OC₁₋₂ haloalkyl;
L¹² is a C₁₋₆ alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and
G² is phenylene, wherein G² is optionally substituted with 1-2 substituents independently selected from halogen, —CN, C₁₋₄ alkyl, C₃₋₄ cycloalkyl, C₁₋₂ haloalkyl, —OC₁₋₄ alkyl, —OC₁₋₂ haloalkyl, —OC₃₋₄ cycloalkyl, or —NO₂; and
(ii) recurring units of formula (II) 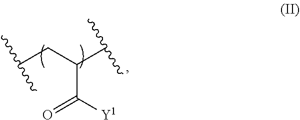
wherein:
Y¹ is —NR^{x}R^{y} or —OR^{x}; and
R^{x} and R^{y} are each independently hydrogen, C₁₋₄ alkyl, or C₃₋₄ cycloalkyl.

2. The polymer of claim 1, wherein B¹ comprises: 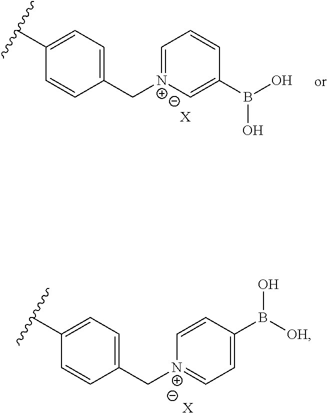
wherein:
X^{Θ} is an anion having a net charge of −1.

3. The polymer of claim 2, wherein X^{Θ} is Br, Cl, NO₃⁻, H₂PO₄⁻, H₂PO₃⁻, HSO₄⁻, HSO₃⁻, H₃C—SO₃⁻, HCO₃⁻, HCO₂⁻, H₃C—CO₂⁻, HC₂O₄₇⁻, or TsO⁻.

4. The polymer of claim 2, wherein X^{Θ} is Br⁻ or Cl⁻.

5. The polymer of claim 1, wherein B¹ comprises:

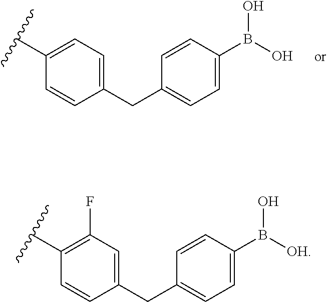 [SMILES: *c1ccc(Cc2ccc(B(O)O)cc2)cc1 ; *c1ccc(Cc2ccc(B(O)O)cc2)cc1F]

6. The polymer of claim 1, wherein L¹ comprises an amide moiety.

7. The polymer of claim 1, wherein L¹ comprises

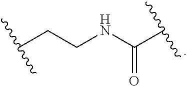 [SMILES: *CCNC(*)=O]

8. The polymer of claim 1, wherein the recurring units of formula (II) are acrylamide units of formula (II-a):

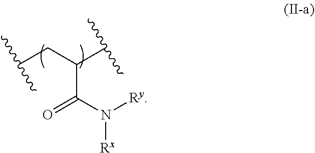

9. The polymer of claim 1, wherein R^{x} and R^{y} are each methyl.

10. The polymer of claim 1, wherein the molar ratio of the units of formula (II) to the units of formula (I) is about 1:1 to about 20:1.

11. The polymer of claim 1, wherein the molar ratio of the units of formula (II) to the units of formula (I) is about 5:1.

12. The polymer of claim 1, wherein the polymer has a number average molecular weight (Mₙ) of about 3,000 g/mol to about 30,000 g/mol as measured by gel permeation chromatography.

13. The polymer of claim 1, wherein the polymer has a Mₙ of about 5,000 g/mol to about 8,500 g/mol as measured by gel permeation chromatography.

14. The polymer of claim 1, wherein the recurring unit of formula (I) is repeated 3 times to 50 times.

15. The polymer of claim 1, wherein the recurring unit of formula (II) is repeated 20 times to 100 times.

16. A hydrogel comprising the polymer of claim 1 crosslinked with a diol crosslinker.

17. The hydrogel of claim 16, wherein the molar ratio of B¹ of the polymer to the diol of the diol crosslinker is from about 0.25:1 to about 10:1.

18. The hydrogel of claim 16, wherein insulin, an insulin variant, an insulin analogue, glucagon, GLP-1, or a combination thereof is encapsulated within the hydrogel.

19. A hydrogel comprising:
the polymer of claim 1 crosslinked with a diol crosslinker including a multi-armed polymer, wherein each individual arm comprises a diol of formula (III):

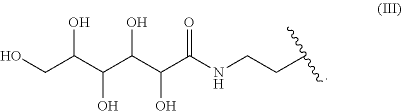 [SMILES: *CCNC(=O)C(O)C(O)C(O)C(O)CO]

20. The hydrogel of claim 19, wherein the molar ratio of B¹ of the polymer to the diol of the diol crosslinker is from about 0.25:1 to about 10:1.

21. The hydrogel of claim 19, wherein the multi-armed polymer comprises a polyalkylene glycol.

22. The hydrogel of claim 21, wherein the multi-armed polymer is a four-armed or an eight-armed polymer.

23. A pharmaceutical composition comprising insulin, an insulin variant, an insulin analogue, glucagon, or GLP-1, or a combination thereof encapsulated within the hydrogel of claim 19, and a pharmaceutically acceptable excipient.

24. A device comprising:
a microneedle array comprising a plurality of microneedles on a surface of a substrate, each microneedle comprising the polymer of claim 1 crosslinked with a diol crosslinker, the diol crosslinker including:
a multi-armed polymer, wherein each individual arm comprises a diol of formula (III): 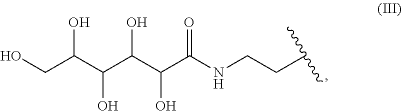 [SMILES: *CCNC(=O)C(O)C(O)C(O)C(O)CO]
wherein the molar ratio of the units of formula (II) to the units of formula (I) is about 1:1 to about 20:1, and the molar ratio of B¹ of the polymer to the diol of the diol crosslinker is about 0.25:1 to about 10:1.

25. The device of claim 24, wherein the diol crosslinker comprises: 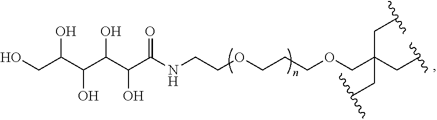
wherein n is 2 to 250.

26. The device of claim 24, wherein the molar ratio of the units of formula (II) to the units of formula (I) is about 5:1.

27. The device of claim 24, wherein the molar ratio of B¹ of the polymer to the diol of the diol crosslinker is about 4:1 to about 8:1.

28. The device of claim 24, wherein each microneedle has a length of about 300 μm to about 800 μm.

29. The device of claim 24, wherein each microneedle lacks a channel extending through the length of the microneedle.

30. The device of claim 24, wherein the substrate comprises the same polymer crosslinked with the diol crosslinker as each microneedle.

31. The device of claim 24, wherein each microneedle has a failure point of greater than 0.6 N/needle.

32. The device of claim 24, wherein each microneedle further comprises insulin, an insulin variant, an insulin analogue, glucagon, GLP-1, or a combination thereof.

33. A method of making a device comprising a microneedle array, the method comprising:
adding the hydrogel of claim 19 to a mold, the mold comprising a plurality of microneedle molds;
applying a force to the hydrogel such that the hydrogel fills each microneedle mold; and
drying the hydrogel to provide a device comprising a microneedle array, the microneedle array comprising a plurality of microneedles that align in number and arrangement with the plurality of microneedle molds, wherein each microneedle comprises the dehydrated hydrogel.

34. The method of claim 33, wherein the insulin, insulin variant, insulin analogue, glucagon, or GLP-1, or combination thereof is encapsulated in the hydrogel.

35. The method of claim 33, wherein the method does not include crosslinking of the hydrogel during or after adding to the mold.

36. A method of delivering insulin to a subject in need thereof, the method comprising:
contacting an area of the subject's skin with the device of claim 24, wherein the insulin, insulin variant, insulin analogue, glucagon, GLP-1, or combination thereof is transdermally delivered to the subject.

37. The method of claim 36, wherein the subject has diabetes.
